(19) United States
(12) Patent Application Publication
(10) Pub. No.: US 2025/0120610 A1
(43) Pub. Date: Apr. 17, 2025

(54) SYSTEM AND METHOD FOR RESPIRATORY RATE MONITORING

(71) Applicant: BIOSIGNS PTE. LTD., Jurong East (SG)

(72) Inventors: Nandakumar Selvaraj, San Jose, CA (US); Nicholas James Huang, Stoneham, MA (US); Menglian Zhou, Palo Alto, CA (US); Pragya Sharma, Jersey City, NJ (US); Dayi Bian, Nashville, TN (US); Pooja Rajiv Mehta, Sunnyvale, CA (US); Milan Mahendra Shah, Hopkinton, MA (US); Kuldeep Singh Vithal Singh Rajput, Boston, MA (US)

(21) Appl. No.: 18/486,846

(22) Filed: Oct. 13, 2023

(51) Int. Cl. A61B 5/08; A61B 5/00

(52) U.S. Cl. CPC A61B 5/0816; A61B 5/7207; A61B 5/7221; A61B 5/7267; A61B 2562/0219

(57) ABSTRACT

Examples provide a system and method for continuous respiratory rate monitoring with greater accuracy and reliability. Sensor data is obtained from a wearable sensor device simultaneously generating both multi-wavelength photoplethysmography (PPG) sensor data as well as inertial measurement unit (IMU) sensor data, such as respiratory body movement data generated by an accelerometer and/or a gyroscope. The system performs signal processing and filtering on the sensor data using a set of rules and threshold(s) to remove Mayer-wave in-band noise, transients, and motion artifacts. PPG-based respiratory rate estimates are generated and combined into a single PPG-based respiratory rate estimate based on the PPG sensor data. IMU-based respiratory rate estimates are generated and combined in parallel using the IMU sensor data to generate a single IMU-based respiratory rate estimate. A final respiratory rate estimate is selected from the final PPG-based respiratory rate estimate and the final IMU-based respiratory rate estimate.

### BACKGROUND

Optical and electro-mechanical forms of physiological sensing have made a plethora of wearable devices useful for health monitoring. Respiratory rate is one of the primary vital signs used to evaluate a person's overall health and respiratory system. Respiratory rate monitoring has a multitude of applications, ranging from personal fitness to medical health monitoring. It is a top predictor of adverse events, and many serious illnesses such as cardiac arrest, COPD, pneumonia, and sepsis. However, accurate continuous estimation of respiratory rate using the wearable devices remain a challenge due to numerous limitations, such as in-band noise from bodily regulatory mechanisms, poor optical strength with some skin tones, motion artifacts, and other limitations. This renders most current techniques of obtaining a patient's respiratory rate impractical and unreliable for use in obtaining continuous respiratory rate measurements due to inconsistent results, and inconvenient for both patients as well as for medical personnel and other caregivers.

### SUMMARY

Some examples provide a system and method for continuous respiratory rate monitoring. A set of sensor devices generates sensor data, including a photoplethysmography (PPG) sensor generating PPG sensor data and inertial movement unit (IMU) sensor generating IMU sensor data associated with a user. A respiratory rate (RR) manager applies a set of rules for filtering and processing the sensor data. The set of rules includes a breath-related peak isolation rule for isolating breath-related peaks from the PPG sensor data and/or a motion activity threshold for identifying respiratory-related movements of the user from the IMU sensor data. The respiratory rate (RR) manager generates respiratory rate estimates by a respiratory rate estimator. The respiratory rate estimates include both PPG-based respiratory rate estimate based on wavelengths of PPG signals extracted from the PPG sensor data and IMU-based respiratory rate estimate based on IMU sensor data in parallel. The RR manager determines a final respiratory rate from one or more respiratory rate estimates based on a multitude of respiratory signal modes, associated quality metrics, a fusion model and/or selection rules. The quality metric includes a quality score for each respiratory rate estimate indicating reliability of a given respiratory rate estimate. The RR manager outputs the final respiratory rate and quality score via a user interface.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

FIG. 1 is an exemplary block diagram illustrating a system for continuous and accurate respiratory rate monitoring.

FIG. 2 is an exemplary block diagram illustrating a respiratory rate (RR) manager for generating a respiratory rate and quality metric using photoplethysmography (PPG) sensor data and inertial measurement unit (IMU) sensor data.

FIG. 3 is an exemplary block diagram illustrating a system for continuous respiratory rate monitoring using optical sensor data and electro-mechanical signals.

FIG. 4 is an exemplary block diagram illustrating a system for processing optical sensor data for use in continuous respiratory rate monitoring.

FIG. 5 is an exemplary block diagram illustrating a pulse respiratory signal processor (PRSP) for processing baseline wander (BW) modulation in the PPG waveform.

FIG. 6 is an exemplary block diagram illustrating a system for processing IMU sensor data for continuous respiratory rate monitoring.

FIG. 7 is an exemplary block diagram illustrating a respiratory signal processor for processing IMU sensor data.

FIG. 8 is an exemplary block diagram illustrating a system for generating respiratory rate estimates using accelerometer (ACC) sensor data.

FIG. 9 is an exemplary flow chart illustrating operation of the computing device to generate a final PPG-based respiratory rate estimate using one or more wavelength PPG signals.

FIG. 10 is an exemplary flow chart illustrating operation of the computing device to generate a respiratory rate and quality using simultaneous multi-wavelength PPG and IMU sensor data.

FIG. 11 is an exemplary flow chart illustrating operation of the computing device to perform continuous respiratory rate monitoring using a set of adaptive rules.

FIG. 12 is an exemplary flow chart illustrating operation of the computing device to process surrogate PPG signals.

FIG. 13 is an exemplary flow chart illustrating operation of the computing device to perform tracking and tracing of respiratory frequency (TTRF).

FIG. 14 is an exemplary line graph illustrating baseline wander (BW) respiratory signal derived from modulations in a PPG signal.

FIG. 15 is an exemplary line graph illustrating amplitude modulation (AM) signal derived from modulations in a PPG signal.

FIG. 16 is an exemplary line graph illustrating frequency modulation (FM) signal derived from modulations in a PPG signal.

FIG. 17 is an exemplary line graph illustrating buffering and interpolation of a surrogate respiratory signal to improve timing accuracy of breath detection.

FIG. 18 is an exemplary line graph illustrating a noise-related peak removed by the adaptive peak-to-trough threshold rather than being identified as a breath.

FIG. 19 is an exemplary line graph illustrating a signal quality-based optimization of the algorithm performance and the outage for the (PPG) pulse respiratory modes.

FIG. 20 is an exemplary line graph illustrating a raw ACC waveform.

FIG. 21 is an exemplary line graph illustrating a ground truth RR signal.

FIG. 22 is an exemplary line graph illustrating ACC respiration waveforms after PCA.

FIG. 23 is an exemplary line graph illustrating motion activity level.

FIG. 24 is an exemplary line graph illustrating Kurtosis-based optimization of algorithm performance and outage for the IMU respiratory modes.

FIG. 25 is an exemplary line graph illustrating a respiration waveform derived from gyroscope sensor data.

FIG. 26 is an exemplary line graph illustrating a respiration waveform derived from ACC sensor data.

FIG. 27 is an exemplary line graph illustrating a reference end-tidal CO₂ waveform from a standard capnograph (CAP) with RR at 5 breaths-per-minute (brpm).

FIG. 28 is an exemplary line graph illustrating a frequency spectrum of gyroscope with a false frequency peak near 10 brpm alongside a true respiratory frequency peak at 5 brpm.

FIG. 29 is an exemplary line graph illustrating a respiration waveform derived from a gyroscope sensor.

FIG. 30 is an exemplary line graph illustrating a respiration waveform derived from ACC sensor data.

FIG. 31 is an exemplary line graph illustrating a reference end-tidal CO₂ waveforms from a standard capnograph (CAP) with RR at 40 brpm.

FIG. 32 is an exemplary line graph illustrating a spectrum of ACC respiration signal with a false frequency peak near 9 brpm alongside a true respiratory frequency peak at 40 brpm.

FIG. 33 is an exemplary line graph illustrating final RR outputs during a sequence of paced breathing tests.

FIG. 34 is an exemplary bar graph illustrating comparison of RR mean absolute error performances among skin tones groups of I-VI for a traditional literature model and the RR manager implemented fusion model.

FIG. 35 is an exemplary line graph illustrating example recording of a subject that illustrates superior performance of the RR manager fusion method compared to an ACC-based subset at high motion activity.

FIG. 36 is an exemplary line graph illustrating motion activity variation over time for a subject.

FIG. 37 is an exemplary correlation scatter graph illustrating clinical validation results for wearable armband devices embedded with the RR manager.

FIG. 38 is an exemplary line graph illustrating a plot showing correlation between the reference CAP RR and the gyroscope-based RR estimate while the user is in an upright position.

FIG. 39 is an exemplary line graph illustrating a correlation between the reference CAP RR and the gyroscope-based RR estimate while the user is in a supine position.

FIG. 40 is an exemplary line graph illustrating correlation between the reference CAP RR and the ACC-based RR estimate while the user is in an upright position.

FIG. 41 is an exemplary line graph illustrating correlation between the reference CAP RR and the ACC-based RR estimate while the user is in the supine position.

FIG. 42 is an exemplary line graph illustrating a plot showing correlation of reference CAP RR with gyroscope-based RR estimate in a low RR range.

FIG. 43 is an exemplary line graph illustrating a plot showing correlation of reference CAP RR with gyroscope-based RR estimate in a normal RR range.

FIG. 44 is an exemplary line graph illustrating a plot showing correlation of reference CAP RR with gyroscope-based RR estimate in a high RR range.

FIG. 45 is an exemplary line graph illustrating a plot showing correlation of reference CAP RR with ACC-based RR estimate in a low RR range.

FIG. 46 is an exemplary line graph illustrating a plot showing correlation of reference CAP RR with ACC-based RR estimate in a normal RR range.

FIG. 47 is an exemplary line graph illustrating a plot showing correlation of reference CAP RR with ACC-based RR estimate in a high RR range.

FIG. 48 is an exemplary table illustrating comparison of the RR manager algorithm with a traditional algorithm.

FIG. 49 illustrates a computing apparatus according to an embodiment as a functional block diagram.

Corresponding reference characters indicate corresponding parts throughout the drawings. Any of the components in the figures may be combined into a single example or embodiment.

### DETAILED DESCRIPTION

A more detailed understanding can be obtained from the following description, presented by way of example, in conjunction with the accompanying drawings. The entities, connections, arrangements, and the like that are depicted in, and in connection with the various figures, are presented by way of example and not by way of limitation. As such, any and all statements or other indications as to what a particular figure depicts, what a particular element or entity in a particular figure is or has, and any and all similar statements, that can in isolation and out of context be read as absolute and therefore limiting, can only properly be read as being constructively preceded by a clause such as “In at least some examples, . . . ” For brevity and clarity of presentation, this implied leading clause is not repeated ad nauseum.

Respiratory rate monitoring can provide valuable and potentially life-saving information to medical providers and other caregivers. The most direct method of measuring respiratory rate is by monitoring the end-tidal carbon dioxide levels directly outside of a user's nose and mouth. However, this method requires cumbersome masks or cannula and is not suitable for long term, unobtrusive continuous measurement especially at out-of-hospital or home settings.

In contrast, optical-based pulse oximeter sensors are ubiquitous in both clinical practice and wearable consumer health devices. They are available in various form-factors which can be attached to a body site, such as a finger, the wrist, the upper arm, and/or the earlobe of a user. These devices allow convenient acquisition of blood volume pulse, also known as photoplethysmography (PPG). PPG signals are modulated by the respiration rate in amplitude, baseline, and frequency. Thus, it is sometimes possible to estimate respiratory rate by extracting these modulations. However, a reliable and accurate respiratory rate estimate from wearable devices for continuous monitoring remains elusive due to limitations, such as in-band low frequency physiological interferences from bodily regulatory mechanisms (e.g., Mayer waves), the Nyquist frequency limit where respiratory signals are aliased when the fundamental cardiac frequency is lower than two times (2X) of the respiratory frequency, lower PPG optical signal strength associated with dark skin tones, and motion artifacts.

For example, the currently used standard or classic PPG-based algorithm for predicting respiratory rate based on a PPG waveform completely fails once the respiratory rate exceeds half the heart rate. In this method, respiration-induced modulations in a PPG waveform are sampled at the pulse peaks with paced breathing at various rates. Due to the Nyquist frequency limit, respiratory surrogate signals are under sampled and aliased when the fundamental heart rate frequency is lower than two times that of the respiratory rate frequency. To overcome these limitations, more sophisticated signal processing steps are necessary to circumvent those specific issues while enhancing the traditional PPG method.

Respiratory rate can also be estimated by measuring periodic movement primarily in the chest wall or secondarily from other core body locations. During the inhalation phase of a respiratory cycle, the diaphragm moves downward, and the chest expands as air enters the lungs; the opposite is true during exhalation. This cyclic movement can be measured using inertial measurement unit (IMU) (e.g., accelerometer and gyroscope) sensors applied on the upper chest via adhesive. A direct form of respiratory waveforms and derived respiratory rate estimates can be obtained by measuring movements of the chest wall during respiration and angular rotations occurring during inhalation and exhalation using these types of sensors. In other example, chest bands utilizing piezo-resistance or capacitance sensing can be used for respiration monitoring, but like direct carbon dioxide (CO₂) measurement methods, these devices are also not suitable and/or comfortable enough for long-term use.

An accelerometer is another example of a wearable device that can measure respiratory induced accelerations of upper body movements when attached to any location on the upper arm for vital sign monitoring, although such a sensor could be attached to any part of the body. Depending upon the sensor attachment site on the body, the signal strengths of respiratory signals measured by the accelerometer can vary drastically. However, if the accelerometer is placed on more peripheral locations away from the upper body, such as on the wrist, finger or digits, the accelerometer's sensitivity to respiratory-related physical movements is attenuated resulting in relatively weak signal with very low signal-to-noise ratio (SNR). For this reason, devices worn on peripheral location, such as the wrist, are typically unable to offer reliable or accurate direct measurement of respiration due to these limitations.

In contrast, referring to the figures, examples of the disclosure enable continuous respiratory rate measurements. In some examples, a wearable device with accelerometer (ACC) sensors is provided that can measure the chest wall movements in a form factor that is more conducive to regular and continuous use. The synthesis of multiple modalities: multi-wavelength PPGs, ACC, and gyroscope, is provided to produce a robust respiratory rate estimate suitable for unobstructive continuous measurement on a wearable form factor device, which can be applied to various parts of a user's body, such as, for example, locations on a person's upper-arm.

Aspects of the disclosure further enable a computational system and method for continuous accurate monitoring of respiratory rate using a wearable sensor device. The system receives simultaneous multi-wavelength optical photoplethysmography (PPG) sensor data and an inertial measurement unit (IMU) sensor data (e.g., from accelerometer and gyroscope) from the wearable sensor device. A respiratory rate manager derives respiratory signals from both the multi-wavelength optical PPG pulse sensor and the IMU sensor of the wearable device. The respiratory signal frequencies are tracked parallelly and independently from the PPG pulse derived respiratory signals and IMU derived respiratory signals using a combination of time-domain, frequency domain and time-frequency spectra-based methodologies. Alongside the respiratory frequency tracking, detection of posture or activity states and movement intensity levels are also determined by a set of independent classifier algorithms by inputting and analyzing the IMU sensor data. The respiratory modes from PPG sensor and IMU sensor are adaptively selected and weighted based on a plurality of conditions including the estimated respiratory frequency band or range and the detected movement levels and posture or activity states. Accordingly, independent estimations of respiratory rates and qualities are further determined for the PPG and IMU sensors and combined finally to achieve a more versatile and robust RR outputs using rule-based selection, machine learning optimization models for quality estimation and error minimization (or performance maximization), or quality weighted averages.

In other examples, an RR manager component analyzes a combination of one or more wavelengths of PPG sensor data and IMU sensor data from an accelerometer and/or gyroscope. The RR manager component may be implemented in software, hardware, firmware, or a combination thereof. The data is processed by the RR manager using a sequence of sophisticated signal processors, involving time-domain and time-frequency domain based adaptive respiratory frequency tracking, and machine learning stages to produce accurate continuous RR outputs from a wearable sensor device. The final RR output is less sensitive to in-band noise signals, Nyquist frequency limits, and skin tones, as well as more robust to the body movements of the user wearing the sensor device.

The computing device operates in an unconventional manner by analyzing sensor data from PPG sensor devices as well as one or more IMU sensor devices simultaneously to produce both PPG-based RR estimates as well as IMU-based RR estimates in parallel. A set of rules are applied to process and filter the signals in order to produce final respiratory rate outputs and associated quality values to indicate the confidence level, thereby improving the functioning of the underlying computing device with more reliability and accuracy.

In still other examples, the system provides an accurate and continuous respiratory rate output via a user interface or other output device which improves user efficiency via the UI interaction while enabling more accurate medical diagnosis and treatment efficacy for improved patient outcomes. A combination of rule-based selections, optimization learning, and machine learning models further enables more accurate and reliable respiratory rate calculations for reduced error rates in respiratory rate measurements.

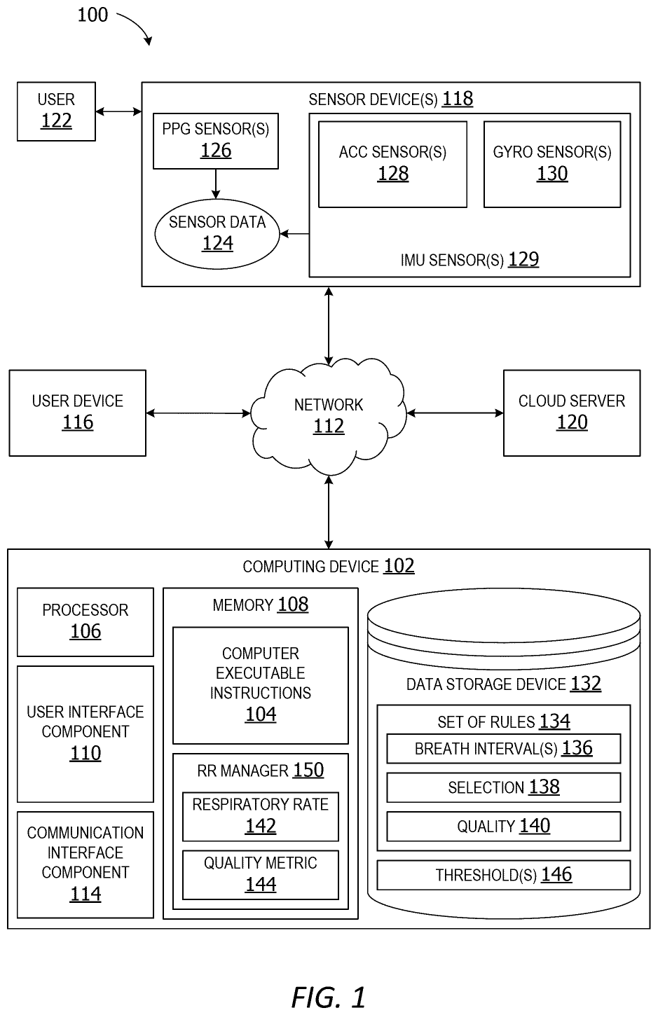

Referring again to FIG. 1, an exemplary block diagram illustrates a system 100 for continuous and accurate respiratory rate (RR) monitoring. In the example of FIG. 1, the computing device 102 represents any device executing computer-executable instructions 104 (e.g., as application programs, operating system functionality, or both) to implement the operations and functionality associated with the computing device 102. The computing device 102, in some examples includes a mobile computing device or any other portable device. A mobile computing device includes, for example but without limitation, a mobile telephone, laptop, tablet, computing pad, netbook, gaming device, wearable computing device, embedded systems, or devices, and/or portable media player. The computing device 102 can also include less-portable devices such as servers, desktop personal computers, kiosks, or tabletop devices. Additionally, the computing device 102 can represent a group of processing units, microcontrollers, microprocessors, or other computing devices.

In some examples, the computing device 102 has at least one processor 106 and a memory 108. The computing device 102, in other examples includes a user interface component 110.

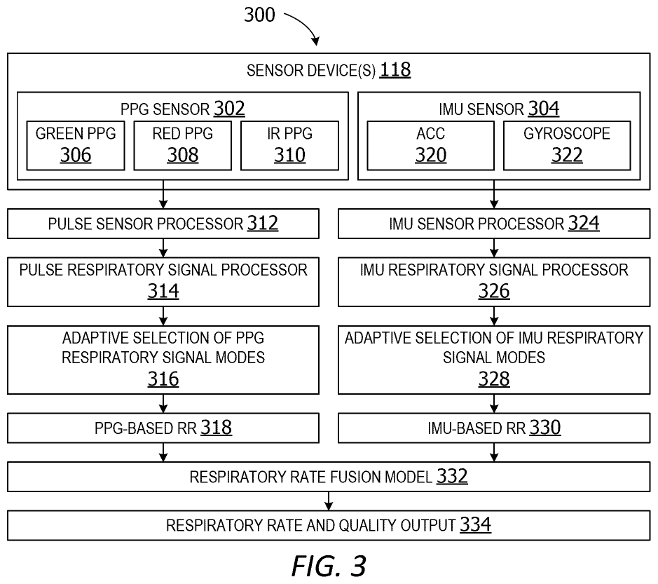

The processor 106 includes any quantity of processing units and is programmed to execute the computer-executable instructions 104. The execution of computer-executable instructions 104 is performed by the processor 106, performed by multiple processors within the computing device 102 or performed by a processor external to the computing device 102. In some examples, the processor 106 is programmed to execute instructions such as those illustrated in the figures (e.g., FIGS. 3-13).

The computing device 102 further has one or more computer-readable media such as the memory 108. The memory 108 includes any quantity of media associated with or accessible by the computing device 102. The memory 108 in these examples is internal to the computing device 102 (as shown in FIG. 1). In other examples, the memory 108 is external to the computing device (not shown) or both (not shown). The memory 108 can include read-only memory and/or memory wired into an analog computing device.

The memory 108 stores data, such as one or more applications. The applications, when executed by the processor 106, operate to perform functionality on the computing device 102. The applications can communicate with counterpart applications or services such as web services accessible via a network 112. In an example, the applications represent downloaded client-side applications that correspond to server-side services executing in a cloud.

In other examples, the user interface component 110 includes a graphics card for displaying data to the user and receiving data from the user. The user interface component 110 can also include computer-executable instructions (e.g., a driver) for operating the graphics card. Further, the user interface component 110 can include a display (e.g., a touch screen display or natural user interface) and/or computer-executable instructions (e.g., a driver) for operating the display. The user interface component 110 can also include one or more of the following to provide data to the user or receive data from the user: speakers, a sound card, a camera, a microphone, a vibration motor, one or more accelerometers, a BLUETOOTH® brand communication module, wireless broadband communication (LTE) module, global positioning system (GPS) hardware, and a photoreceptive light sensor. In a non-limiting example, the user inputs commands or manipulates data by moving the computing device 102 in one or more ways.

The network 112 is implemented by one or more physical network components, such as, but without limitation, routers, switches, network interface cards (NICs), and other network devices. The network 112 is any type of network for enabling communications with remote computing devices, such as, but not limited to, a local area network (LAN), a subnet, a wide area network (WAN), a wireless (Wi-Fi) network, or any other type of network. In this example, the network 112 is a WAN, such as the Internet. However, in other examples, the network 112 is a local or private LAN.

In some examples, the system 100 optionally includes a communications interface component 114. The communications interface component 114 includes a network interface card and/or computer-executable instructions (e.g., a driver) for operating the network interface card. Communication between the computing device 102 and other devices, such as but not limited to a user device 116, one or more sensor device(s) 118 and/or a cloud server 120, can occur using any protocol or mechanism over any wired or wireless connection. In some examples, the communications interface component 114 is operable with short range communication technologies such as by using near-field communication (NFC) tags.

The user device 116 represents any device executing computer-executable instructions. The user device 116 can be implemented as a mobile computing device, such as, but not limited to, a wearable computing device, a mobile telephone, laptop, tablet, computing pad, netbook, gaming device, and/or any other portable device. The user device 116 includes at least one processor and a memory. The user device 116 can optionally also include a user interface component.

The one or more sensor device(s) 118 include sensor devices for generating sensor data 124 associated with a user 122. In some examples, the sensor device(s) 118 include one or more sensors, such as a photoplethysmography (PPG) sensor 126, accelerometer (ACC) sensor 128, and/or gyroscope (Gyro) sensor 130. The sensor device(s) 118 in this example are wearable sensor devices, such as sensor devices in a watch, arm band, chest band, adhesive patch, or other wearable device form factors.

The cloud server 120 is a logical server providing services to the computing device 102 or other clients, such as, but not limited to, the user device 116. The cloud server 120 is hosted and/or delivered via the network 112. In some non-limiting examples, the cloud server 120 is associated with one or more physical servers in one or more data centers. In other examples, the cloud server 120 is associated with a distributed network of servers.

The system 100 can optionally include a data storage device 132 for storing data, such as, but not limited to a set of rules 134. The set of rules 134 includes one or more rules for isolating breath interval(s) 136 in sensor data 124, one or more rules for selection 138 of a respiratory rate 142, one or more rules for determining quality 140 of a calculated respiratory rate 142 using a quality metric 144 and/or one or more threshold(s) 146 for determining respiratory rates.

For example, the set of rules 134 can be used to isolate the most reliable breath intervals from which to calculate a respiration rate. In some examples, breath intervals are required to pass one or more of these tests to be included in the average used during RR calculations. One rule in the set of rules 134 requires that the peak-to-trough amplitude must be greater than a percentage of the previous breaths. In another example, a rule in the set of rules 134 states a breath duration should be within a range of desired duration compared to a predetermined set of previous breath intervals to be included. In another example rule, motion activity around the interval must be less than a predetermined threshold value to be included. The set of rules 134 are applied to PPG sensor data to ensure that only peaks in the surrogate respiratory signal that are caused by the breathing cycle of the user 122 are identified as breath intervals and that other smaller peaks caused by random fluctuations in the signal, outliers in the data, transients, and motion artifacts are disregarded for more reliable and accurate RR calculations. Aside from the motion activity threshold, these limits are adjusted based on the recent history of breath-related peaks. This enables the system to adapt in real-time to varying noise situations and breathing patterns of the user 122.

The data storage device 132 can include one or more different types of data storage devices, such as, for example, one or more rotating disks drives, one or more solid state drives (SSDs), and/or any other type of data storage device. The data storage device 132 in some non-limiting examples includes a redundant array of independent disks (RAID) array. In some non-limiting examples, the data storage device(s) provide a shared data store accessible by two or more hosts in a cluster. For example, the data storage device may include a hard disk, a redundant array of independent disks (RAID), a flash memory drive, a storage area network (SAN), or other data storage device. In other examples, the data storage device 132 includes a database.

The data storage device 132 in this example is included within the computing device 102, attached to the computing device, plugged into the computing device, or otherwise associated with the computing device 102. In other examples, the data storage device 132 includes a remote data storage accessed by the computing device via the network 112, such as a remote data storage device, a data storage in a remote data center, or a cloud storage.

The memory 108 in some examples stores one or more computer-executable components. Exemplary components include a respiratory rate (RR) manager 150. In some examples, the RR manager 150 receives sensor data generated by the wearable sensor device(s) 118. A wearable sensor device in the set of one or more sensor device(s) 118 is applied to any location on the body of the user 122.

The sensor device(s) 118 measures optical signals, such as measurement of blood volume using one or more wavelengths of PPG and IMU signals 129 including one or more axes of accelerometer and gyroscope signals. The optical pulse and movement related IMU signals are each input to a specified sequence of additional signal conditioning and signal processing stages to produce independent estimates of the respiratory rate 142. The respiratory rate may also be referred to as the respiration rate or breathing rate.

The RR manager 150 derives a plurality of respiratory signals from optical PPG sensor data (green, red, and infrared) and IMU sensor data (accelerometer and gyroscope). The RR manager 150 adaptively tracks respiratory frequencies on a time- and time-frequency domain approaches, respectively. These simultaneous RR frequency estimates are combined to obtain one final estimate of RR with a quality estimate. Finally, the derived RR values are outputted and displayed at suitable interfaces of the system, such as, but not limited to, the user interface component 110 and/or a user interface of the user device 116.

In this example, the RR manager 150 calculates the respiratory rate and quality metric on the computing device 102. However, in other examples, the RR manager 150 resides on the cloud server 120. In this example, the sensor data 124 is transmitted to the cloud server. The RR manager on the cloud server calculates the respiratory rate 142. The cloud server transmits the respiratory rate and quality metric to the computing device via the network.

In the example of FIG. 1, the set of rules 134 is stored on the data storage device on the computing device 102. In other examples, the set of rules 134 is stored on a cloud storage and accessed by the computing device via the network 112.

Likewise, in the example of FIG. 1, the set of sensor device(s) is shown as a separate device or devices from the computing device 102. However, in other examples, the computing device 102 is a wearable computing device, such as arm band, which includes both the RR manager implemented on the processor 106 as well as the sensor device(s) included within the same computing device 102. In this example, the wearable computing device includes the sensor devices generating the sensor data as well as the software RR manager component for generating the RR estimates using the sensor data. In these examples, the final RR and quality metric value can be displayed on the user interface component 110 or transmitted to the user device for presentation to the user.

In other examples, the RR manager 150 extracts pulse-related peaks in the PPG signal using a peak-finding method. The RR manager extracts modulations of these pulse-related peaks in terms of amplitude (AM), frequency (FM), and baseline (BW) to obtain three surrogate respiratory signals. For each of these estimates of the respiratory signal, filtering is used to remove noises such as Mayer waves and motion artifacts. After filtering, breaths are identified through peak finding with various outlier, transient, and motion rejection rules to filter out peaks which are not related by breaths. Information from previous RR estimates and/or other sensor modalities is used to track RR more accurately. For example, information such as accelerometer and gyroscope data may be used. Finally, an RR estimate is obtained for each modulation and combined using a RR fusion method.

The examples of FIG. 1 are described above in the context of determining RR using a wearable sensor device or sensor devices attached to the user 122, such as a patient. These examples are illustrative and not intended to be limited by the context. Various modifications to the disclosed embodiments, the principles or the features described herein will be readily apparent to those skilled in the art. The following descriptions of individual processes are provided in the context of patent application and its requirements. Thus, the present disclosure is not intended to limit the invention to the embodiments shown; rather, the invention is to be accorded the widest scope consistent with the principles and features described herein.

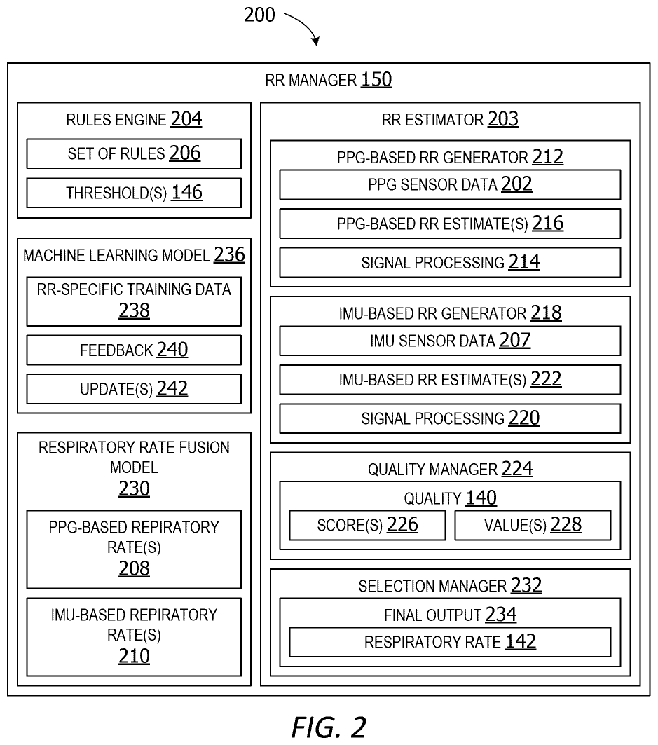

FIG. 2 is an exemplary block diagram illustrating a respiratory rate manager 150 for generating a respiratory rate 142 and quality 140 metric using PPG sensor data 202 and IMU sensor data 207. In some examples, a rules engine 204 is a software component which maintains, stores, and/or updates a set of rules 206 for isolating breath intervals from PPG sensor data 202, selecting respiratory rate 142 from a PPG-based respiratory rate 208 and an IMU-based respiratory rate 210. In some examples, the rules engine 204 optionally also includes one or more threshold(s) 146 used during the processing of PPG and IMU sensor data.

RR estimator 203 is a software, hardware, or firmware component that generates respiratory rate estimates based on sensor data generated by the wearable sensor devices. In this example, the RR estimator 203 includes a PPG-based respiratory rate generator 212 and an IMU-based respiratory rate generator 218. The PPG-based respiratory rate generator 212 analyzes PPG sensor data 202 using a sequence of signal processing 214 steps to generate one or more PPG-based respiratory rate estimate(s) 216.

An IMU-based respiratory rate generator 218 simultaneously applies IMU signal processing 220 to IMU sensor data to generate one or more IMU-based respiratory rate estimate(s) 222. In other words, one or more IMU-based respiratory rate estimates and one or more PPG-based respiratory rate estimates are generated at substantially the same time and in parallel.

A quality manager 224 component analyzes the PPG-based RR estimate(s) 216 and the IMU-based RR estimate(s) 222 to determine a quality 140 level of the estimates. The quality manager 224 generates a quality metric, such as one or more quality value(s) 228 and/or one or more score(s) 226. If the quality of a given RR estimate falls below a minimum threshold, the RR estimate is discarded or disregarded.

In some examples, a respiratory rate fusion model 230 combines two or more PPG-based RR estimate(s) 216 into a single, final PPG-based RR estimate. The respiratory rate fusion model 230 likewise combines two or more IMU-based RR estimate(s) 222 into a single, final IMU-based respiratory rate. A selection manager 232 analyzes the PPG-based RR estimate and the IMU-based RR estimate. The selection manager selects a final respiratory rate 142 for output based on the quality 140 of the estimates. The final output 234 in some examples includes both the final respiratory rate and the quality metric value or quality score for the selected respiratory rate.

In some examples, the RR manager 150 includes a machine learning (ML) model 236. The ML model 236, in some examples, includes one or more performance optimized model(s). The ML model 236 may include pattern recognition, modeling, or other machine learning algorithms to analyze sensor data and/or database information to update the rules in the set of rules 206, update the threshold(s) 146, generate alerts, including notifications and/or instructions. In this example, the ML model 236 is trained using RR-specific training data 238. In other examples, the ML model 236 is trained using feedback provided by one or more users. The ML model update(s) 242 the set of rules 206 and/or the threshold(s) 146 as the ML model learns using the training data and/or the feedback 240.

FIG. 3 is an exemplary block diagram illustrating a system 300 for continuous respiratory rate monitoring using optical based PPG sensor data and electro-mechanical based inertial measurement unit (IMU) sensor data. The system 300 is a system for generating respiratory rates using data from a wearable sensor device, such as, but not limited to, the system 100 in FIG. 1.

The system 300 provides parallel processing pathways for the various optical sensor data generated by PPG sensor(s) 302 and the electro-mechanical signals generated by IMU sensor(s) 304. Multiple wavelengths of PPG signals are processed by first identifying the heartbeat related pulses and then extracting various estimates of the respiratory signal. Breaths are identified as peaks in these time-domain signals, with a plethora of outlier, transient, motion, and noise rejection processes. Optical-based estimates are derived from the corresponding inter-breath intervals and are combined into an intermediate RR measurement through an adaptive selection process.

In this example, the wavelengths of PPG signals include green PPG 306, red PPG 308 and/or infrared (IR) PPG 310. The PPG signals are processed by a pulse sensor processor 312 and a pulse respiratory signal processor 314 to filter and process the PPG signals. The RR manager performs adaptive selection of PPG respiratory signal modes 316. The RR manager generates PPG-based RR 318 and a quality metric indicating quality of the generated PPG-based RR 318.

At the same time, accelerometer 320 and gyroscope 322 data are used to estimate RR from movement in the chest wall. These signals are pre-processed by IMU sensor processor 324 to segment the data into windows with minimal non-respiratory movements. The time-frequency spectrum information is extracted from the signal and respiratory frequency tracking is performed by the IMU respiratory signal processor 326 to follow the respiratory rate signal through time. The RR manager adaptively selects the IMU respiratory signal modes 328. As with the optical estimates, these IMU-based RR estimates 330 are combined through adaptive selection of signal modes and weights accounting the estimated respiratory rates, posture states, and movement levels. Finally, a respiratory rate fusion model 332 combines the RR measurements from the two modalities to produce one final output 334. The combination of the signal modalities as well as the juxtaposition of time-domain and frequency-domain processing work together to ensure the reliability of the final respiratory rate and quality estimate.

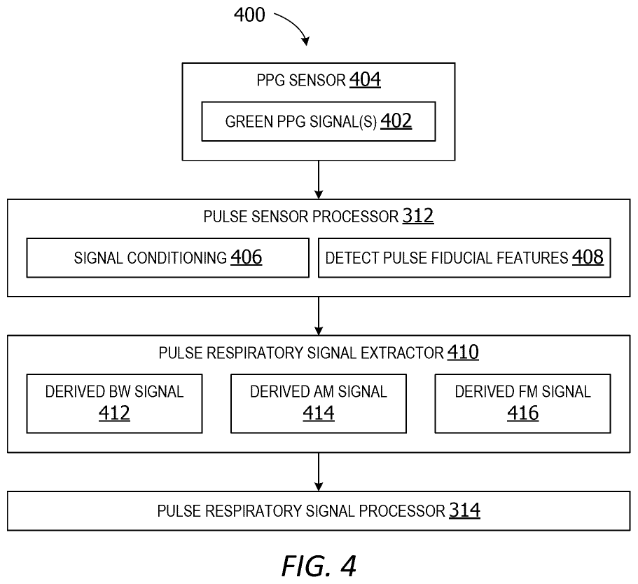

Turning now to FIG. 4, an exemplary block diagram illustrating a system 400 for processing optical sensor data for use in continuous respiratory rate monitoring. One or more wavelengths of PPG generated by a PPG sensor are processed by the RR manager to generate a respiratory rate and quality metric value indicating the quality and predicted accuracy of the calculated respiratory rate. In this example, the PPG signal includes one or more green wavelengths of PPG signal(s) 402 generated by at least one PPG sensor 404, such as, but not limited to, a sensor in the set of sensor device(s) 118 in FIG. 1 and/or the PPG sensor 302 in FIG. 3. The pulse sensor processor 312 performs signal conditioning 406 and detects pulse fiducial features 408 of the PPG signal(s).

A pulse respiratory signal extractor (PRSE) 410 extracts a plurality of respiratory waveform signals modulated by the respiratory function of the human body from the PPG signal(s). The extracted pulse respiratory signals include derived baseline wander (BW) signal 412, derived amplitude modulation (AM) signal 414, and/or derived frequency modulation (FM) signal 416.

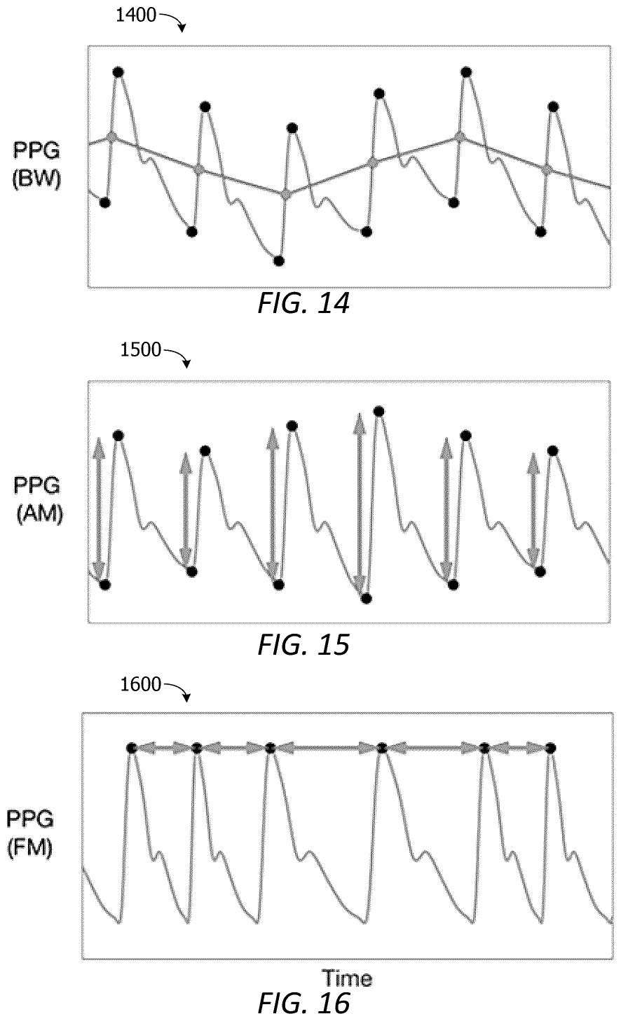

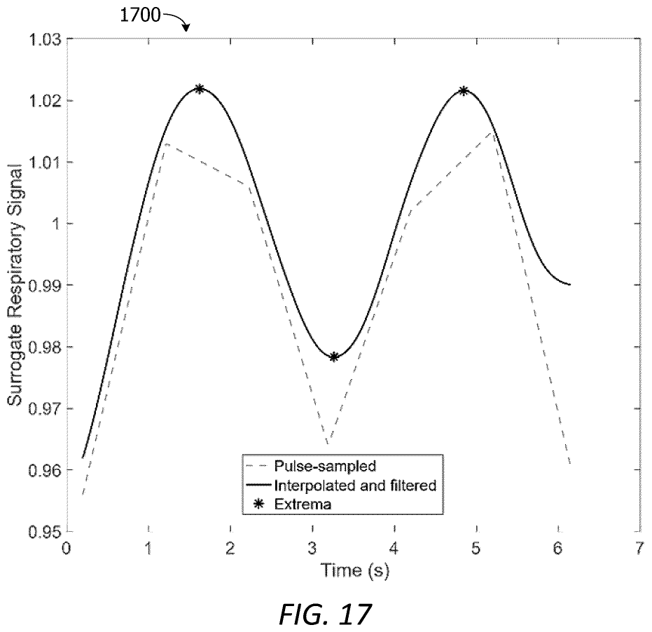

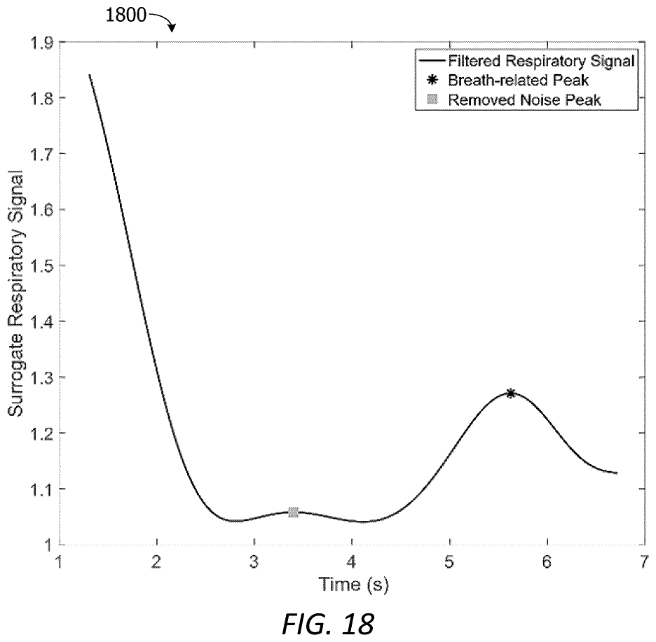

The PPG-based RR estimation procedure builds on the method described in the background. Conceptually, estimates of the respiratory signal are extracted from various modulations in the PPG waveform including the AM, FM, and BW. However, additional filtering, adaptive thresholds, outlier removal, and additional smoothing procedures are all used to reduce the effects of noise and to improve the consistency of the RR estimate as in FIG. 17, and FIG. 18. The BW signal, AM signal and FM signal are discussed in greater detail in FIG. 14, FIG. 15, and FIG. 16 below.

The extracted plurality of pulse respiratory signals is processed by one or more pulse respiratory signal processor (PRSP) 314. The PRSP 314 performs a sequence of signal processing, including adaptive filtering and tracking of periodic respiratory peaks, rejecting of in-band Mayer waves, suppressing of high-frequency transients, and removing motion artifacts.

Mayer waves are low frequency (˜0.1 Hz or 6 breaths per minute) naturally occurring oscillations in arterial blood pressure. Without a method of removal, they can be dominating in-band noise signals that interfere with RR estimation. An example of a PPG spectrum with such an overpowering Mayer wave signal can occur as a strong consistent energy between 5-10 breath rate per minute (brpm). The influence of Mayer waves is particularly powerful in green wavelength PPG signals, as discussed in FIG. 4. Although the SNR for other wavelengths, such as Infrared and Red PPG waveforms, is somewhat lower than for the green wavelength, they are relatively less influenced by the Mayer waves. Thus, the inclusion of additional PPG wavelengths serves to mitigate the effects of in-band noise physiological signals such as Mayer waves.

Despite the sophisticated signal conditioning and adaptive controls incorporated in the PPG sensor-based RR pathway towards in-band Mayer-wave and motion rejections, the sensitivity and accuracy of PPG sensor data for RR estimation are still limited because measuring respiration from PPG modulations is indirect, and those modulations can also be influenced by autonomic and other control mechanisms of the human body. Though the signal strength of respiratory signal embedded in accelerometer and/or gyroscope measurements from upper body can be feeble or weak, they can produce accurate RR measurements at least during stationary also known as rest conditions as a direct form of respiration via transduction of respiratory body movements with appropriate signal processing tools and controls. The RR manager addresses this issue and overcomes the limitations with the proposed simultaneous method of time-frequency spectra-based RR tracking from IMU signals including ACC motion signal. Then, RR outputs from IMU and RR estimations from PPG and their respective qualities are compared to one or more set of rules at the final stages of the RR algorithm, producing the overall final RR output for the given segment time.

Although the example of FIG. 4 refers to green wavelengths of PPG signal(s), the examples are not limited to only green wavelengths. In other examples, the system includes red and infrared wavelengths of PPG signal(s), as well as any other wavelengths or types of PPG signal(s).

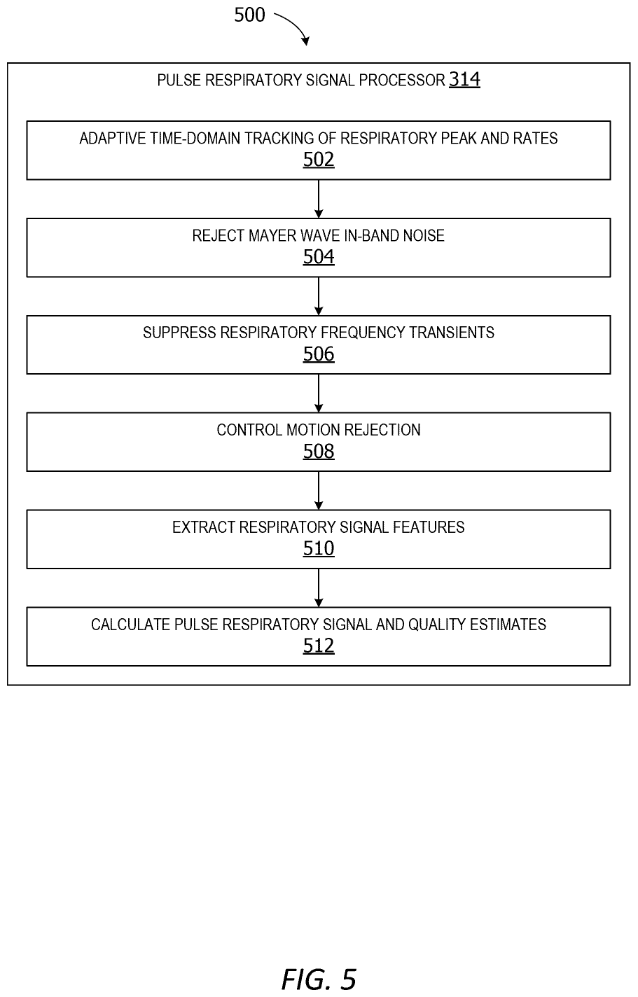

FIG. 5 is an exemplary block diagram illustrating a pulse respiratory signal processor (PRSP) for processing respiration-induced modulation in the PPG waveform. In some examples, the PRSP 314 performs a sequence of signal processing on the pulse respiratory signal modes derived from the PPG signal, such as the BW signals, AM signals and/or FM signals. The signal processing includes adaptive time-domain tracking of respiratory peak and rates at 502. The PRSP rejects Mayer wave in-band noise at 504. The PRSP suppresses respiratory frequency transients at 506. The PRSP 314 controls motion rejection by removing motion artifacts at 508. The PRSP 314 extracts respiratory signal features at 510 and calculates independent respiratory rate estimated values and quality estimates from one or more of the pulse respiratory signal modes at 512. In some examples, the selection and fusion of PPG channels and pulse respiratory signal modes are automated based on pulse respiratory features, RR values, and quality estimates producing an optical-sensor-based intermediate respiratory rate(s) and quality output(s).

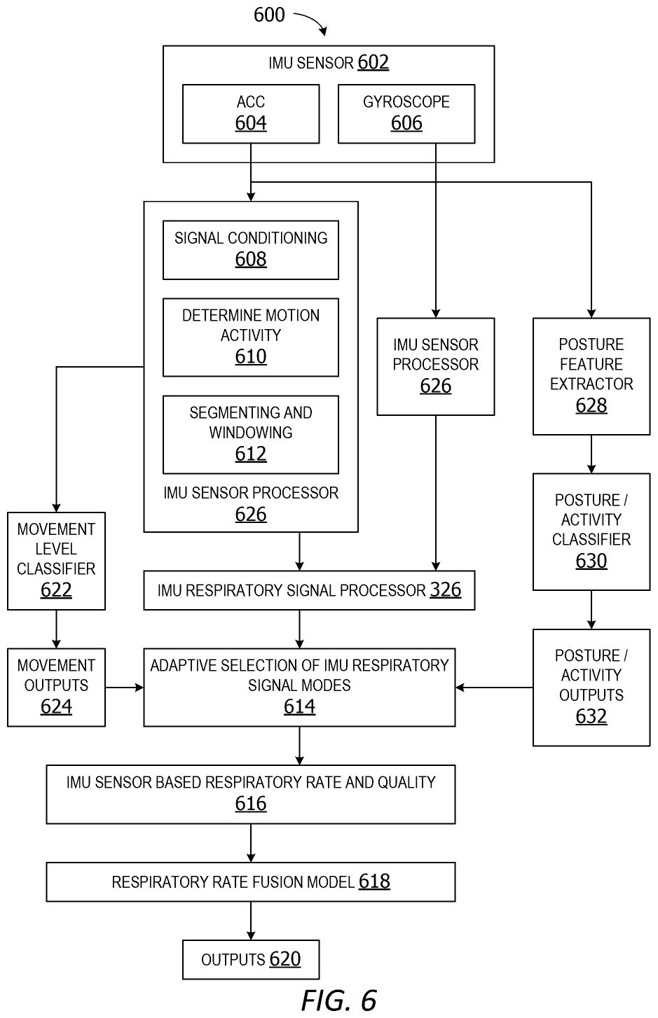

FIG. 6 is an exemplary block diagram illustrating a system 600 for processing IMU sensor data for continuous respiratory rate monitoring. In some examples, one or more axes of accelerometer 604 and/or gyroscope 606 of IMU sensor 602 are processed by one or more IMU sensor processor 324. The input IMU signals are conditioned 608, segmented and windowed 612 for resting stationary and or ambulatory conditions based on the detected motion activity levels 610. An IMU respiratory signal processor (IMURSP) 326 extracts the respiration-induced upper body movements from the processed and segmented accelerometer and/or gyroscope signals. In the IMU respiratory signal processor 326, plurality of accelerometer or gyroscope axes are combined into a principle respiratory component (PRC) and the time-varying variance of PRC is determined simultaneously. In one example, principal component analysis (PCA) is employed for axes fusion, then tracks the RR trace from the time-frequency spectrum of the fused principal respiratory component (PRC) and evaluates the quality of the RR estimate using the variance or latent of PRC. The selection of IMU respiratory signal modes 614 and fusion of IMU signal channels and IMU respiratory signal modes are automated based on IMU respiratory features, approximated RR ranges, concurrent motion activity or movement levels, posture conditions and signal quality estimates producing an IMU sensor based intermediate RR and quality outputs 616. The independent RR and quality estimates from optical and IMU sensors are inputted to a respiratory rate fusion model 618 producing the final RR and quality output 620.

For example, the respiratory rate fusion model can be any of a mathematical, statistical, heuristic or regression models. For example, a linear or nonlinear combination or a decision tree of rules applied to independent qualities and RR produce the final outputs of RR and corresponding quality estimates.

In some examples, a movement level classifier 622 generates movement outputs 624 associated with the movement data obtained via analysis of the IMU sensor data. The adaptive selection of IMU respiratory signal modes 614 is optionally performed using the movement outputs in addition to the respiratory signals and quality values.

In still other examples, data generated by the gyroscope is processed by an IMU sensor processor 626 in parallel with accelerometer data processed by the IMU sensor processor 626. The IMU sensor processor 626 is a processor, such as, but not limited to, the IMU sensor processor 324 in FIG. 3. The outputs of the IMU sensor processor 626 include processed gyroscope signal data associated with movements of the user. The outputs are utilized by the IMURSP 326 to select IMU respiratory signal modes associated with respiration of the user for utilization in calculating the RR of the user.

A posture feature extractor 628 in other examples extracts data associated with a posture of the user's body during respiration. A posture/activity classifier 630 generates posture/activity outputs 632 describing the activity level and/or posture of the user's body based on accelerometer and gyroscope IMU sensor data. The posture and/or activity outputs 632 are utilized by the RR manager to adaptively select the highest quality RR estimates for use in calculating and/or selecting the final respiratory rate of the user.

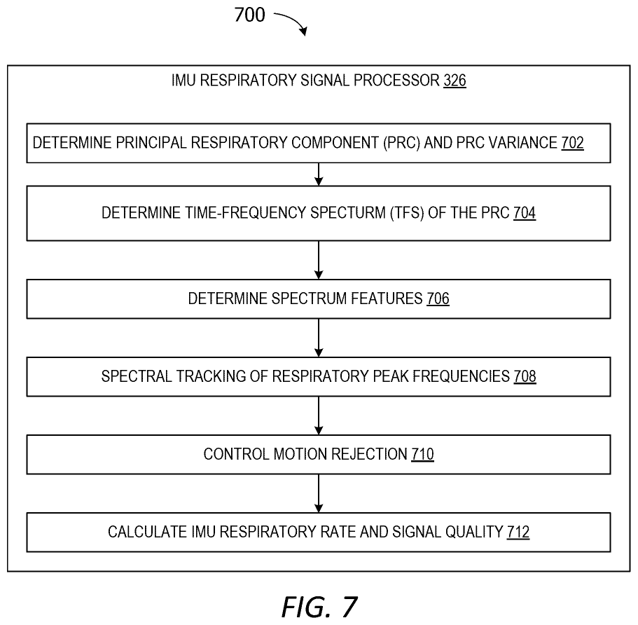

FIG. 7 is an exemplary block diagram illustrating an IMU respiratory signal processor 326 for processing IMU sensor data. In some examples, the IMU respiratory signal processor 326 combines a plurality of accelerometer or gyroscope axes into a principle respiratory component (PRC) and the time-varying variance of PRC is determined simultaneously at 702.

In one example, principal component analysis (PCA) is employed for axes fusion, then the RR trace to determine the time-frequency spectrum (TFS) of the fused principal respiratory component (PRC) is tracked at 704. The IMURSP determines spectrum features at 706. The IMURSP performs tracking and tracing of respiratory frequencies at 708 and controlling of motion rejection at 710. The IMURSP evaluates the quality of the RR estimate using the variance or latent of PRC.

IMU respiratory rate and signal quality is calculated at 712. In this manner, the selection and fusion of IMU signal channels and IMU respiratory signal modes are automated based on IMU respiratory features, approximated RR ranges, concurrent motion activity or movement levels, posture conditions and signal quality estimates producing an IMU sensor based intermediate RR and quality outputs.

In some examples, the independent RR and quality estimates from optical and IMU sensors are inputted to a respiratory rate fusion model producing the final RR and quality output. For example, the respiratory rate fusion model can be any of a mathematical, statistical, heuristic or regression models. For an example, a linear or nonlinear combination or a decision tree of rules applied to independent qualities and RR produce the final outputs of RR and corresponding quality estimates.

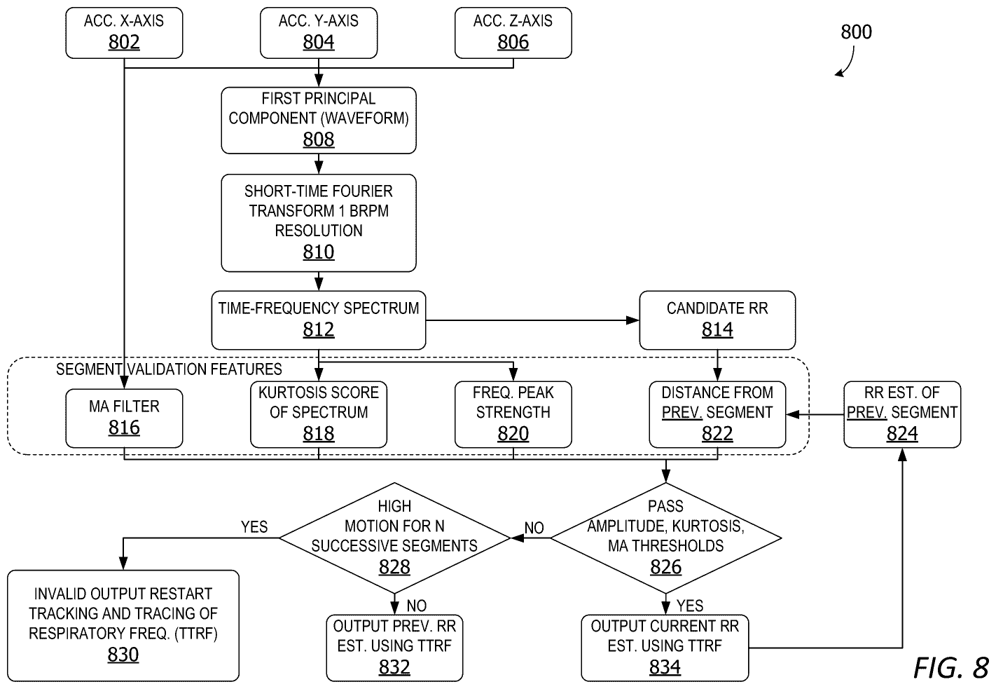

FIG. 8 is an exemplary block diagram illustrating a system 800 for generating respiratory rate estimates using accelerometer (ACC) sensor data. The system 800 is a system for generating IMU-based RR and quality estimates, such as, but not limited to, the system 100 in FIG. 1, the system 300 in FIG. 3, and/or the system 600 in FIG. 6. An instantiation of the ACC-based RR estimation procedure is shown in FIG. 8.

In this example, acceleration is recorded in three orthogonal directions, including the ACC x-axis 802, the ACC y-axis 804, and the ACC z-axis 806. Principal component analysis (PCA) is used to extract the respiratory induced motion waveform 808 from these three ACC waveforms, and the time-frequency spectrum 812 is calculated using the Short-time Fourier transform (STFT) at 810.

A candidate respiratory rate 814 is filtered using segment validation algorithms to determine its validity, such as, but not limited to, motion activity filter 816, kurtosis score of spectrum 818, frequency peak strength 820, and/or distance from the previous segment 822. For example, the candidate RR may only be considered if the motion activity is below a certain threshold to avoid motion artifacts, and if the amplitude at the respiration frequency is above another threshold to ensure that the respiratory signal is strongly present. Additionally, the candidate RR should not vary too far from the previously estimated RR value, which would require the respiratory rate estimate of previous valid segment 824 to be included.

A determination is made whether to pass amplitude, kurtosis, and/or MA thresholds at 826. If yes, the current respiratory rate estimate is output using tracking and tracing of respiratory frequency (TTRF) 834. Where high motion for “n” successive segments 828 is present, the output is invalid and TTRF is restarted at 830. If high motion is not present at 828, the previous respiratory rate estimation is output using TTRF at 832.

In some examples, the respiration rate is extracted through a TTRF method 834 on this spectrum, with motion activity, Kurtosis of the spectrum, and amplitude of the candidate respiratory frequency peak 826 helping to determine the TTRF based RR estimate. When the RR estimate using current spectrum is determined less reliable, the previous RR estimate is held for up to a preset time duration, after which no estimate is produced, and the TTRF is reset 830. In one example, the latent of the first principal component is used for evaluating the quality of the RR estimate given that the respiration induced movement has limited amplitude hence higher latent value indicates the existence of interference motion.

In other examples, the final RR outputs are determined for each segment based on one or more set of rules that take the quality estimations of PPG and ACC methods and accordingly switches between PPG-based RR estimates and ACC-based RR estimates, such as, but not limited to, the set of rules 134 in FIG. 1. In this example, if the ACC-based estimate has quality higher than a preset quality threshold, such as 65%, the RR estimate from ACC method is output for the given segment. Otherwise, PPG-based estimate is the final output.

In another example, the ACC method is validated based on spectral features, such as kurtosis. When it is reliable, it can also be used to guide the PPG estimate. Since the ACC-based method has relatively stringent requirements for motion activity level, it has higher outage than the PPG-based method, and the PPG-based estimate will be used during the ACC-based estimate outage.

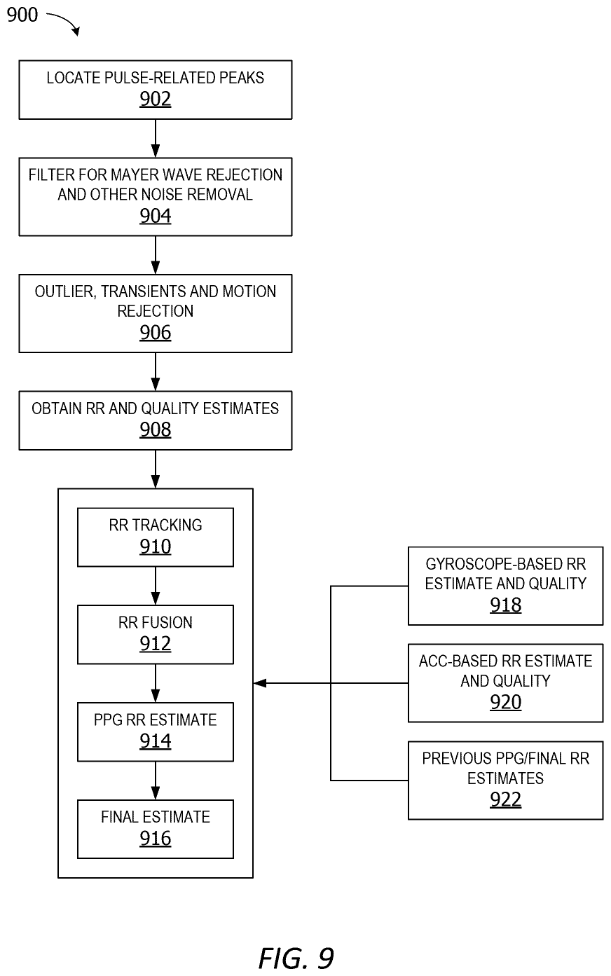

FIG. 9 is an exemplary flow chart 900 illustrating operation of the computing device to generate a final PPG-based respiratory rate estimate using one or more wavelength PPG signals. The process shown in FIG. 9 is performed by an RR manager component, executing on a computing device, such as the computing device 102 in FIG. 1.

The RR manager obtains one or more wavelengths of PPG sensor data and locates pulse-related (systolic) peaks in the PPG signal(s) at 902. Additionally, other PPG waveform fiducial markers such as (diastolic) troughs or max slope events from each cardiac cycle can also be located to derive surrogate respiratory signals. The RR manager performs signal processing to eliminate signal noise. The RR manager filters the signal(s) for Mayer wave rejection and other noise removal at 904. The RR manager performs outlier, transient and motion rejection at 906. The RR manager obtains respiratory rate and quality estimates at 908.

The RR manager performs respiratory rate tracking at 910. In some examples, parallelly processed gyroscope-based RR and quality estimations at 918 is used in conjunction with the PPG-based RR 914 and quality estimations. The ACC-based RR estimates and quality 920 and/or previous PPG/final estimates at 922 may also be used for RR tracking at 910. The RR manager performs RR fusion at 912 of the plurality of PPG-based RR estimates, using inputs like quality and current and/or previous RR to get the PPG estimate at 914. The RR manager determines the final RR estimate at 922 from the parallel PPG/ACC/Gyroscope-based RR estimations and their associated quality values. These RR estimates from the previous segments may also be used for the current segment with all the processing stages: RR tracking 910, RR fusion 912, PPG RR estimate 914 and final RR estimate 916.

While the operations illustrated in FIG. 9 are performed by a computing device, aspects of the disclosure contemplate performance of the operations by other entities. In a non-limiting example, a cloud service performs one or more of the operations. In another example, one or more computer-readable storage media storing computer-readable instructions may execute to cause at least one processor to implement the operations illustrated in FIG. 9.

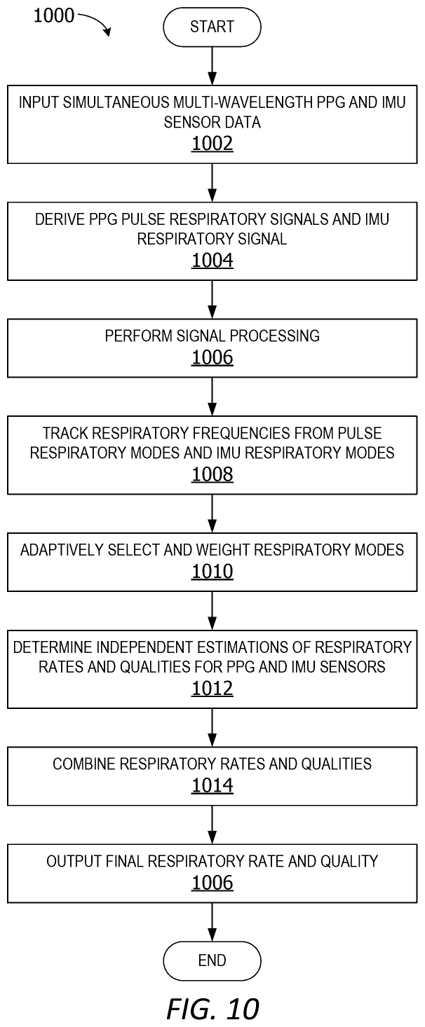

FIG. 10 is an exemplary flow chart 1000 illustrating operation of the computing device to generate a respiratory rate and quality using simultaneous multi-wavelength PPG and IMU sensor data. The process shown in FIG. 10 is performed by an RR manager component, executing on a computing device, such as the computing device 102 in FIG. 1.

The process begins with the RR manager receiving simultaneous multi-wavelength PPG sensor data and IMU sensor data at 1002. The IMU sensor data includes one or more axes of accelerometer and/or gyroscope signals. The optical pulse PPG signals and movement related IMU signals are each input to the RR manager. The RR manager derives PPG pulse respiratory signals from optical PPG sensor data and IMU respiratory signals at 1004. The IMU respiratory signals include a time-frequency spectra from IMU signals. The RR manager performs signal processing at 1006. The signal processing includes a specified sequence of additional signal conditioning and signal processing stages to produce independent estimates of the respiration rate. The RR manager adaptively tracks respiratory frequencies from pulse respiratory modes and IMU respiratory modes on a time- and time-frequency domain approaches, respectively at 1008. The RR manager adaptively selects and weights the respiratory modes at 1010. The RR manager determines independent estimations of respiratory rates and qualities for PPG and IMU sensors at 1012. The RR manager combines the simultaneous respiratory rates and qualities at 1014. The frequency estimates are combined to obtain one final estimate of RR with a quality estimate. Finally, the derived RR values are output at 916. The output includes the final respiratory rate and quality.

While the operations illustrated in FIG. 10 are performed by a computing device, aspects of the disclosure contemplate performance of the operations by other entities. In a non-limiting example, a cloud service performs one or more of the operations. In another example, one or more computer-readable storage media storing computer-readable instructions may execute to cause at least one processor to implement the operations illustrated in FIG. 10.

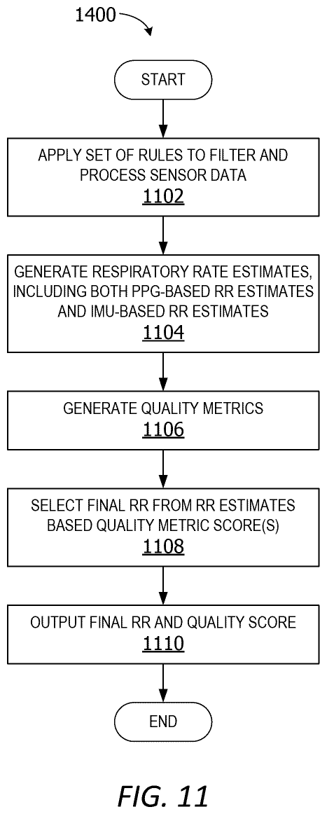

FIG. 11 is an exemplary flow chart 1100 illustrating operation of the computing device to perform continuous respiratory rate monitoring using a set of adaptive rules. The process shown in FIG. 11 is performed by an RR manager component, executing on a computing device, such as the computing device 102 in FIG. 1.

The process begins by applying a set of rules to filter and process sensor data at 1102. The RR manager generates respiratory rate estimates, including both PPG-based RR estimates and IMU-based RR estimates at 1104. The RR manager generates quality metrics at 1106. The quality metrics include a quality score or other quality value for each RR estimate. The RR manager selects a final RR from the RR estimates based on the quality metric score(s) for each RR estimate at 1108. The final RR and quality score for the final RR are output at 1110.

While the operations illustrated in FIG. 11 are performed by a computing device, aspects of the disclosure contemplate performance of the operations by other entities. In a non-limiting example, a cloud service performs one or more of the operations. In another example, one or more computer-readable storage media storing computer-readable instructions may execute to cause at least one processor to implement the operations illustrated in FIG. 11.

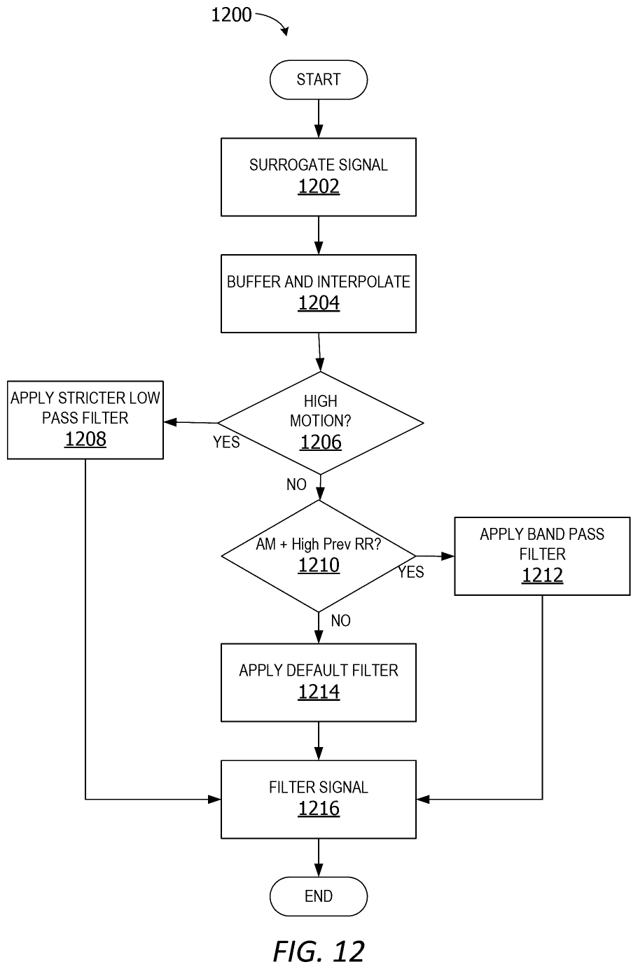

FIG. 12 is an exemplary flow chart 1200 illustrating operation of the computing device to process surrogate PPG-derived respiratory signals. The process shown in FIG. 12 is performed by an RR manager component, executing on a computing device, such as the computing device 102 in FIG. 1. The process in FIG. 12 is an exemplary noise filtering block. The process in FIG. 12 describes transient filtering and motion rejection controls of PPG-based RR estimation.

The process begins with receiving a surrogate PPG derived respiratory signal such as amplitude modulation, frequency modulation or baseline wander at 1202. These surrogate respiratory signals are sampled only at pulse-related peaks in the PPG signal at any wavelengths of green, red, or infrared. The surrogate respiratory signal is buffered and interpolated at 1204 to achieve a uniform sampling rate, for example at a rate of 4 Hz. A filter is chosen to further isolate or decompose only the respiration frequency band; the characteristics of the filter used are very important and are chosen adaptively based on the specific modulation signal used, the presence of motion, and the relative changes in previous RR estimate.

A determination is made whether there is high motion activity at 1206. If yes, a strict low pass filter is applied based on the uniformly sampled surrogate respiratory signal to reject high frequency noise and artifacts produced due to motion and improve composure of the surrogate respiratory signal with true respiration band of frequencies. If the signal is not in motion, a determination is made whether the signal is amplitude modulation with high previous respiratory rate at 1210. If not, a default filter is applied at 1214 to filter the signal. The default filtering strategy is a low pass filter at a fixed cutoff frequency, LP1 (e.g., 42 brpm). If the surrogate signal is based on amplitude modulation at 1210, a bandpass filter is applied to reduce the effects of low frequency Mayer waves, but only if the previous RR estimate is high (making it likely that the true respiratory frequency band and the Mayer waves are separable). The signal is filtered at 1216 by using the low pass filter at 1208, the default filter at 1214 and/or the band pass filter at 1212. The process terminates thereafter.

While the operations illustrated in FIG. 12 are performed by a computing device, aspects of the disclosure contemplate performance of the operations by other entities. In a non-limiting example, a cloud service performs one or more of the operations. In another example, one or more computer-readable storage media storing computer-readable instructions may execute to cause at least one processor to implement the operations illustrated in FIG. 12.

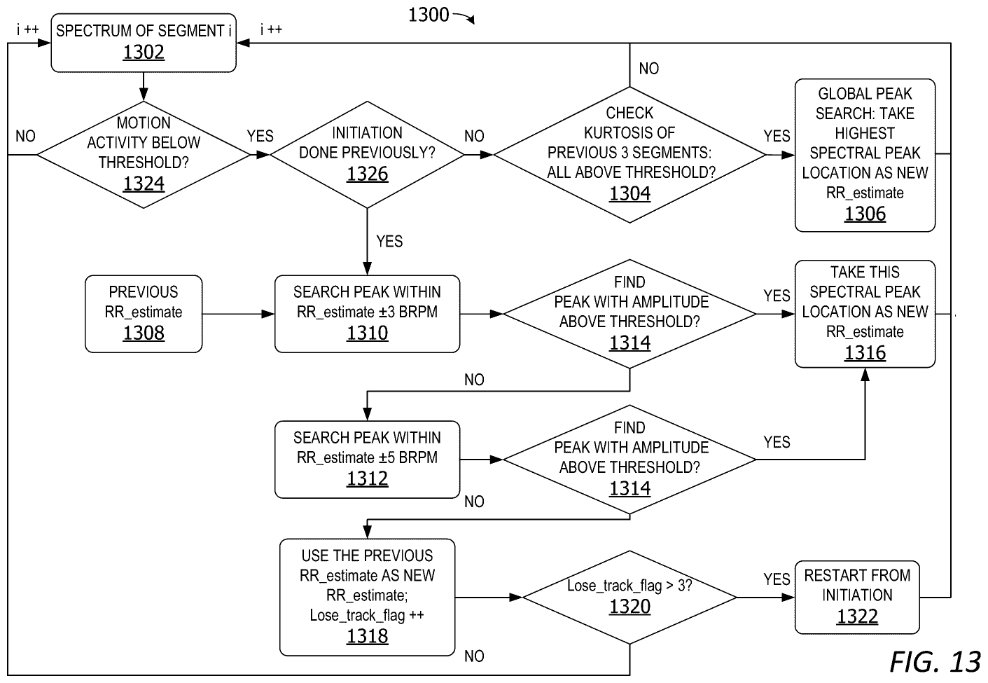

FIG. 13 is an exemplary diagram 1300 illustrating tracking and tracing of respiratory frequency (TTRF) of segment(s) 1302. As shown in FIG. 13, the RR manager initiates TTRF once three consecutive segments' spectral kurtosis score exceeds kurtosis threshold (higher kurtosis indicates more dominating spectral peak) at 1304: the maximum spectral peak location is considered the first RR estimate at 1306. Thus, the first RR estimate at 1306 is now the previous RR_estimate at 1308.

For the next segment, if there is a spectral peak close to the last segment's peak location (first search within +3 brpm 1310 then search within +5 brpm 1312) and this peak amplitude is above the amplitude threshold at 1314, this peak location is used as RR estimate 1316. If no valid spectral peak can be found, the last segment's RR estimate is used at 1318. If more than three consecutive segments have no valid spectral peak at 1320, the current TTRF is terminated, and the process restarts from initiation at 1322. If the motion activity is below threshold at 1324 and initiation is done previously at 1326, the system searches for peaks within the RR-estimate ±3 brpm at 1310. The TTRF restarts once three consecutive segments' spectral kurtosis score exceeds kurtosis threshold again at 1304.

In some examples, the surrogate respiratory signals can be derived from modulations in the PPG signal, as shown in FIG. 14, FIG. 15, and FIG. 16. Referring to FIG. 14, an exemplary line graph 1400 illustrating baseline wander (BW) respiratory signal derived from baseline modulations in a PPG signal. The maximum upward slope of systolic cardiac cycles are the fiducial waveform points/events used for the derivation of baseline wander respiratory signal. In other examples, the diastolic troughs or systolic peaks can also be used as fiducial points/events to derive the baseline wander respiratory signal. FIG. 15 is an exemplary line graph 1500 illustrating exemplary amplitude modulation (AM) signal derived from modulations of pulse amplitudes between diastolic troughs and systolic peak fiducial events of a PPG signal. FIG. 16 is an exemplary line graph 1600 illustrating exemplary frequency modulation (FM) signal derived from modulations of inter-pulse-intervals (IPI) determined as the time duration between successive systolic peak fiducial events of a PPG signal. In other examples, the diastolic troughs or maximum upward slope events can also be used as fiducial points to derive the inter-pulse-intervals as the surrogate respiratory signal containing frequency modulations.

FIG. 17 is an exemplary line graph 1700 illustrating exemplary interpolation and filtering of a surrogate respiratory signal to improve timing accuracy of breath detection. The graph 1700 exhibits the benefits of the interpolation and filtering step through an example of a surrogate respiratory signal derived from PPG data. The respiratory signal is sampled relatively coarsely at the pulse peaks in the PPG data, and as such the breath-related peaks are not directly sampled. By applying interpolation and filtering, a smoother respiratory estimate is obtained, with greater accuracy in terms of timing to the breath identification.

Following the filtering and identification of breath-related peaks in the surrogate respiratory signals, one or more rules are applied by the RR manager component to isolate the most reliable breath intervals for use in calculating a respiration rate from PPG sensor data. The detected breath intervals are required to pass one or more tests to be included in calculating the RR. For example, a rule in the set of rules requires the trough to peak amplitude must be greater than a percentage of the previous breaths. In another example, a rule specifies that the breath duration must be within a range of desired duration compared to a predetermined set of previous breath intervals. In another example, the rule requires motion activity around the interval must be less than a predetermined threshold value. These criteria ensure that only peaks in the surrogate respiratory signal that are caused by the breathing cycle are identified, as opposed to peaks due to fluctuations in the signal induced by other physiological mechanisms, outliers, transients, and motion artifacts. Aside from the motion activity threshold, these limits are adjusted based on the recent history of breath-related peaks, in order to adapt to varying noise situations and breathing patterns.

FIG. 18 is an exemplary line graph 1800 illustrating an exemplary noise-related peak removed by the adaptive trough-to-peak amplitude threshold rather than being identified as a breath. An example of such a case where a small noise-related peak is disregarded in the breath identification process can be seen in Error! Reference source not found. The small peak designated by the gray square is removed and does not influence the final RR estimate because the small peak is likely not caused by the breathing cycle but rather due to random fluctuations in the signal.

Once breath-related peaks are isolated from the surrogate respiratory signals, they are converted into a respiration rate by taking the reciprocal of the average peak-to-peak inter-breath interval (IBRI) in a window. From the independent RR estimates made from each surrogate signal, a quality metric is calculated. This quality metric may be based on such information as the coefficient of variation amongst inter-breath-intervals or inter-breath amplitudes (CVIBRI and CVAmp respectively) and the number of valid breaths (n). The quality metric is calculated in one example based on Error! Reference source not found.:

$$RR Quality = {( \frac{1}{C {V}_{IBRI} * C {V}_{A m p}} )}^{2} * {log}_{2} n \qquad ( 1 )$$

This quality metric is used to determine which estimates are the most reliable in order to perform a quality-based fusion of the RR estimations. The accelerometer-based estimate may be incorporated into the quality fusion, or it may be combined with the final PPG-based estimate. Transients are further reduced by taking median estimated respiration rate over the last certain duration such as a 15 second duration. The quality metric is used to control the tradeoff between the algorithm performance and the outage of RR output by setting different quality thresholds to invalidate low-quality RR estimates.

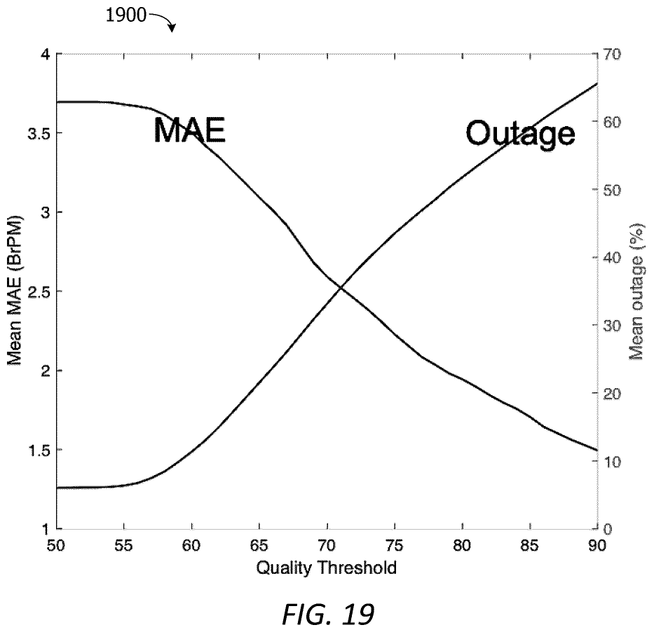

FIG. 19 is an exemplary line graph 1900 illustrating an exemplary signal quality-based optimization of the algorithm mean absolute error (MAE) performance and the outage (i.e., 100%-coverage) for the (PPG) pulse respiratory modes.

The graph 1900 demonstrates an example of how a quality metric and associated threshold can affect the algorithm's performance and outage. As shown in FIG. 19, the higher the quality threshold, the better performance the algorithm can achieve, with the tradeoff of higher RR outage. The system allows the RR manager to optimize the quality, performance, and outage characteristics of continuous RR measurements.

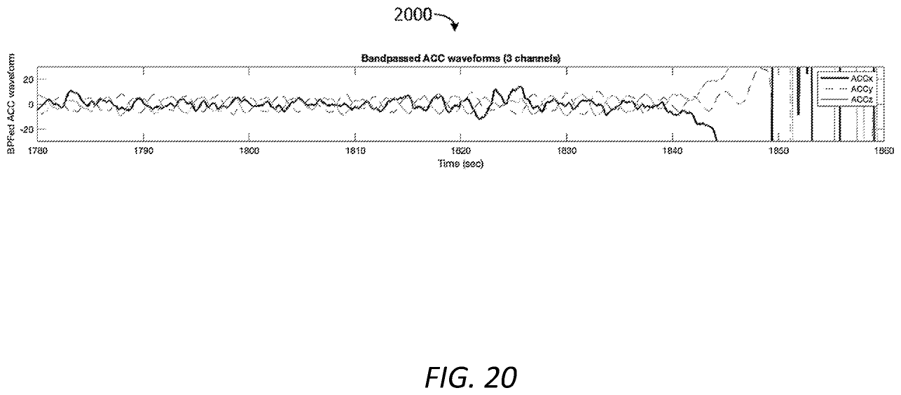

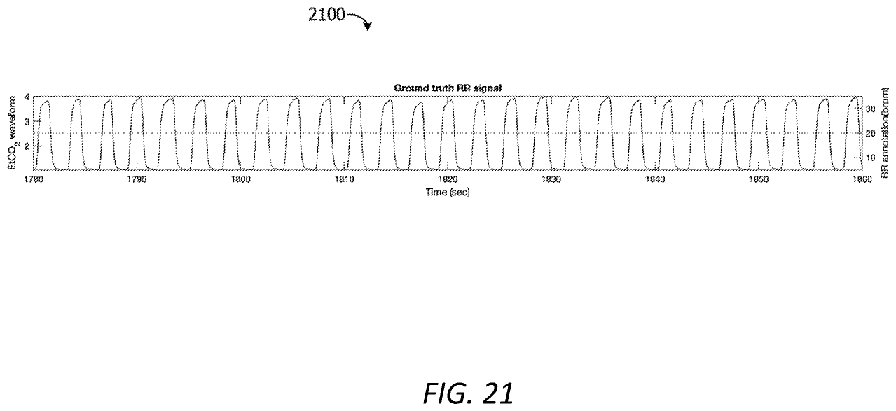

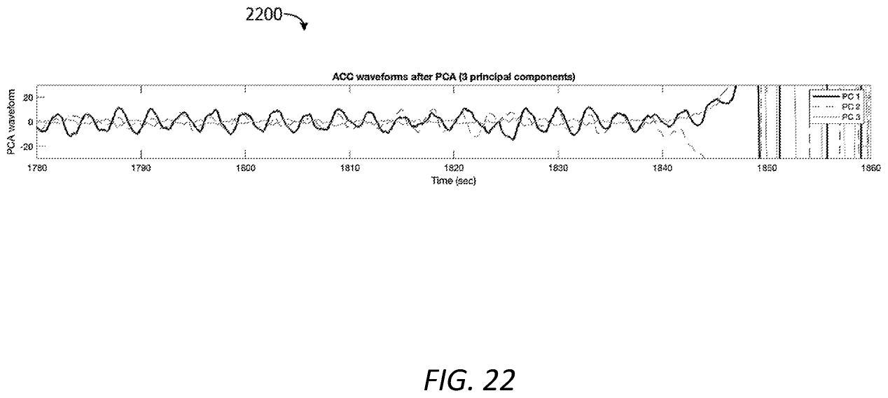

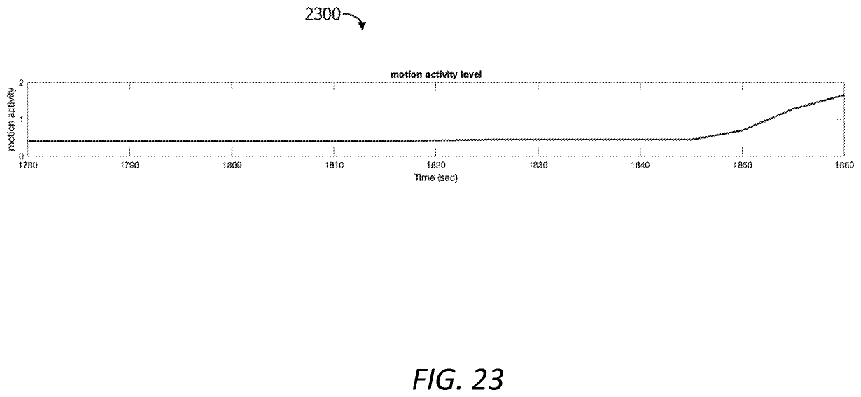

Referring now to FIG. 20, an exemplary line graph 2000 illustrating raw ACC waveform is shown. FIG. 21 is an exemplary line graph 2100 illustrating end-tidal CO₂ waveform (EtCO₂) as a ground truth RR signal. FIG. 22 is an exemplary line graph 2200 illustrating ACC respiration waveforms after PCA. FIG. 23 is an exemplary line graph 2300 illustrating motion activity level.

In some examples, there are three orthogonal accelerometers utilized which contain some degree of the respiration induced by chest wall and upper body movements, where the respiratory frequency matches with a reference End-Tidal Carbon Dioxide (EtCO₂) waveform. However, since the angle between the user's arm and the user's chest varies from time to time, the dominant axis with peak respiratory amplitudes also changes dynamically depending on numerous factors including body postures. To extract respiratory induced motion and associated periodic fluctuations, the bandpass filter is applied to the accelerometer waveform, cutting-off at the frequency range of interest, then PCA is applied on the three accelerometer waveforms to find out the direction with maximum variance, which is the chest motion direction as long as the motion activity level is low. After PCA, the first principal component, PC1, is used, in one example, for time-frequency spectrum analysis. If the averaging motion activity (MA) level (derived from accelerometer signal for every one second, in accordance with the following equation as an example) of a segment is above MA threshold, only partial waveform (where the MA is below threshold) is used for PCA analysis, in accordance with the Equation 2:

$$motion activity = polyfit ( \sqrt{mean ( Δ {acc}_{x}^{2} + Δ {acc}_{y}^{2} + Δ {acc}_{z}^{2} )} ) \qquad ( 2 )$$

In other examples, motion activity is derived with any of linear or nonlinear mathematical transformations applied to the absolute amplitude, relative amplitude, variance changes over one or more axes of accelerations.

When comparing the time-frequency spectra, the PC 1 waveform has more dominating RR trace and lower noise background than the three accelerometer waveforms, including the ACC x-axis (ACCx), ACC y-axis (ACCy) and the ACC z-axis (ACCz). The spectrum of the PC 3 waveform barely contains any RR trace, indicating effective separation of the noise and interference motion from the respiration related motion which benefits the next tracking and tracing of respiratory frequency (TTRF) step. In an example of extraction of the respiratory rate signal from three separate axes of accelerometer using principal component analysis, the stepwise accelerometer trace becomes markedly clearer after the projection.

In an example of the normalized time-frequency spectra (window size 30 sec, step size 5 sec) of a PPG signal and a PCA-combined ACC signal recorded during a paced breathing study, the PPG signal spectrum is dominated by heart rate and Mayer wave components, at around 50 bpm and 5 bpm, respectively. In contrast, the ACC signal spectrum has a strong respiratory peak (except the transition periods between paced breathing sessions, where either the motion activity or the kurtosis score is high), which decreases and increases in a stepwise manner.

The example embodiments of the disclosed method for determining respiratory rate using a combination of optical and electro-mechanical sensor data can be implemented entirely on any of suitable hardware including, but not limited to, bedside or portable monitors, and embedded systems on a variety of form factors such as arm band, wrist bands, watches, adhesive sensors using any or a combination of suitable microcontroller, system on chip, processor, single to multicore central processing unit. The instructions or the program codes of the method are executed as a firmware of the hardware element. The library of the processing methods can be stored on any of suitable memory unit, memory storage secured memory card or cartridge, computer readable medium, volatile, and non-volatile memory in any of the forms of semiconductor, electronic, magnetic, optical systems. The disclosed method can also be implemented as a complete software solution including but not limited to a firmware library, an application software or application programmable interface.

In one example, the system is deployed as an API on the mobile application or the web browser of a computing device. The proposed approach can also combine software and hardware elements, where the API or the software library can be deployed and integrated with the hardware solution producing and displaying respiratory rate estimations on the user interfaces of the hardware solution.

In this example, the hardware elements are including, but not limited to, the mobile smartphones, tablets, bedside monitors, relays, wall mounted hardware units, Internet of Things (IOT) devices, edge computing devices, and powerful wearable embedded systems comprised of microprocessor, volatile or non-volatile memory units such as read-only memory, random access memory, distributed memory storages, and secured memory cards or cartridges, and display units.

The quality of an accelerometer-based RR estimate can be evaluated using a variety of features, such as the latent of the principal component analysis and/or the kurtosis of the combined time-frequency spectrum. The kurtosis would identify how strong the RR peak is relative to the energy at other frequencies and is strongly correlated to the accuracy of the estimate.

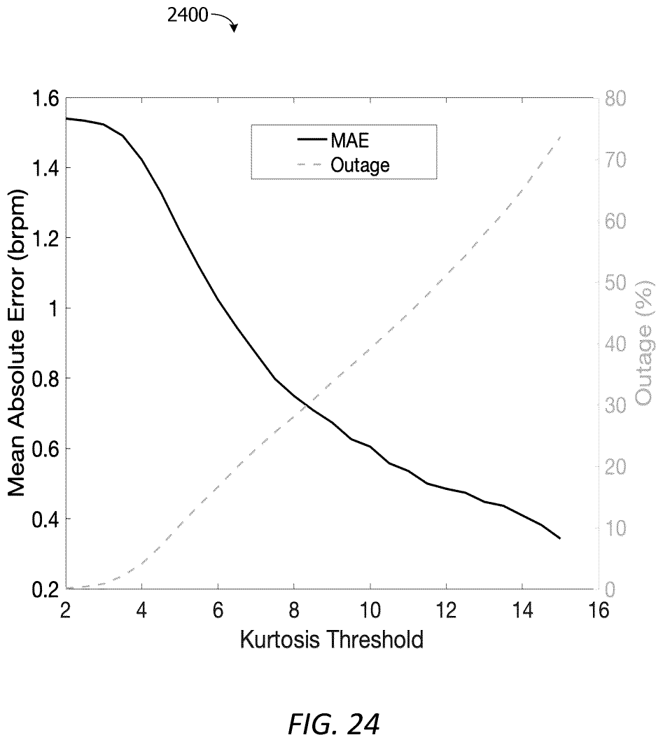

FIG. 24 is an exemplary line graph 2400 illustrating Kurtosis-based optimization of algorithm performance and outage for the IMU respiratory modes. As can be seen in FIG. 24, the mean absolute error of the ACC-based estimate drops sharply as the kurtosis threshold increases.

Gyroscope (Gyro) sensor device(s) capture respiratory motion similar to ACC, and RR derived from both can be fused together. The Gyro measures angular velocity in three directions, roll, pitch, and yaw (GyroR, GyroP, GyroY), and adds to the degrees of motion captured by the IMU sensor, that also measures linear acceleration from ACC. Thus, appropriate fusion of both can improve the RR estimation and coverage with various posture and device placement changes.

The RR estimation from gyroscope (Gyro) sensors is similar to the process for processing data generated by an accelerometer, with ACCx, ACCy and ACCz components replaced by GyroR, GyroP and GyroY to derive respiratory waveforms which are combined using PCA. While motion activity derived from gyroscope sensor is a scaled version of ACC-derived MA, for consistent threshold use, ACC-derived motion activity can be used for processing of both ACC and Gyro respiration signals.

Gyroscope respiratory waveforms are observed to have dominant harmonic components at second or third harmonic of the RR, particularly at low RR, which can potentially have higher power than the fundamental RR, resulting in incorrect RR estimates from the respiratory frequency tracking algorithm. Hence, an additional harmonic correction is performed after peak detection: reject the frequency with maximum peak (fₘₐₓ₁), if second-highest peak exists at a lower frequency (fₘₐₓ₂) (that can be constrained to be a harmonic) and spectrum power ratio of both is greater than a threshold, T∈[0.5, 1), as shown in Equation 3:

$$IF ( {f}_{max 2} < {f}_{max 1} ) \& ( \frac{P ( {f}_{max 2} )}{P ( {f}_{max 1} )} > T ) : RR = {f}_{max 2} \qquad ( 3 )$$

$$Else : RR = {f}_{max 1}$$

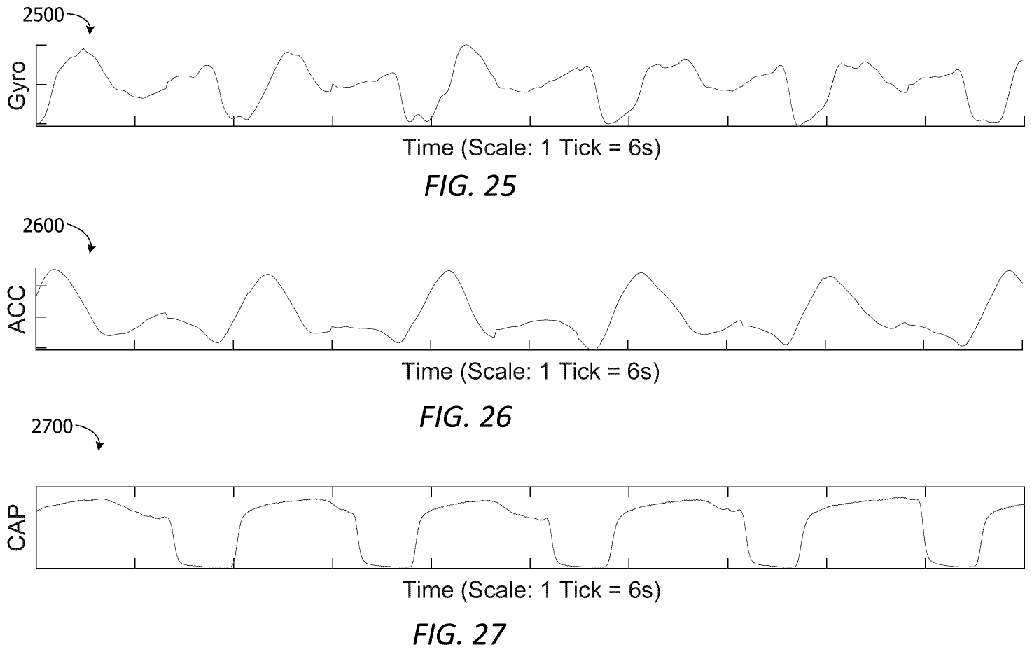

FIG. 25 is an exemplary line graph 2500 illustrating a respiration waveform derived from gyroscope (GYRO) sensor data. The horizontal axis (x-axis) shows changes over time where one tick equals six seconds (6 s). FIG. 26 is an exemplary line graph 2600 illustrating a respiration waveform derived from ACC sensor data. The horizontal axis (x-axis) shows changes over time where one tick equals six seconds (6 s). FIG. 27 is an exemplary line graph 2700 illustrating a reference end-tidal CO₂ waveform from a standard capnography (CAP) with RR at 5 brpm. The horizontal axis (x-axis) shows changes over time where one tick equals six seconds (6 s).

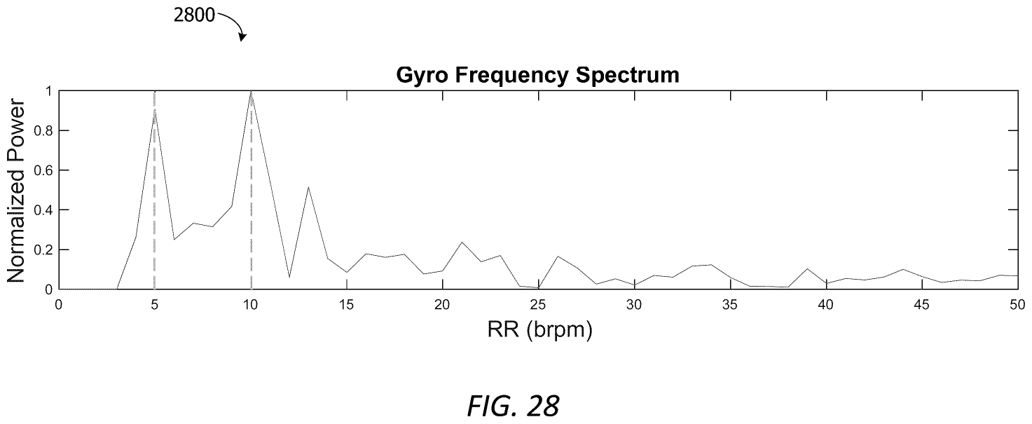

FIG. 28 is an exemplary line graph 2800 illustrating a gyroscope spectrum with a false frequency peak near 10 brpm alongside a true respiratory frequency peak at 5 brpm. This gyroscope frequency spectrum graph includes a vertical axis (y-axis) showing normalized power. The horizontal axis (x-axis) shows respiratory rate (RR) in breaths-per-minute (brpm).

FIG. 25-FIG. 28 discussed above show an example case with gyroscope and accelerometer derived respiration waveform after PCA. CAP reference waveform shows RR=5 brpm. Gyroscope spectrum is shown in FIG. 28 with peak frequency, fₘₐₓ₁=10 brpm and second peak at fₘₐₓ₂=5 brpm. Harmonic correction accurately results in RR of 5 brpm, after rejecting peak at 10 brpm. Since this behavior is more dominant at low RR, the correction can be restricted to peak frequencies detected in range 10-20 brpm.

As accelerometer and gyroscope devices capture respiratory motion differently, their RR performance varies over different postures and RR-ranges. Study under posture variation shows superior gyroscope performance compared to ACC in all postures, particularly in upright posture, which generally has higher RR estimation error. Thus, a higher weightage to gyroscope RR estimate in upright posture can improve RR estimation performance. Further, gyroscope outperforms ACC at high RR range (>20 brpm), and similar to ACC at normal RR (12-20 brpm). At low RR (<12 brpm) ACC estimates are more accurate, with harmonic correction reducing the gap between ACC and gyroscope performance as shown in FIG. 25, FIG. 26, FIG. 27, and FIG. 28.

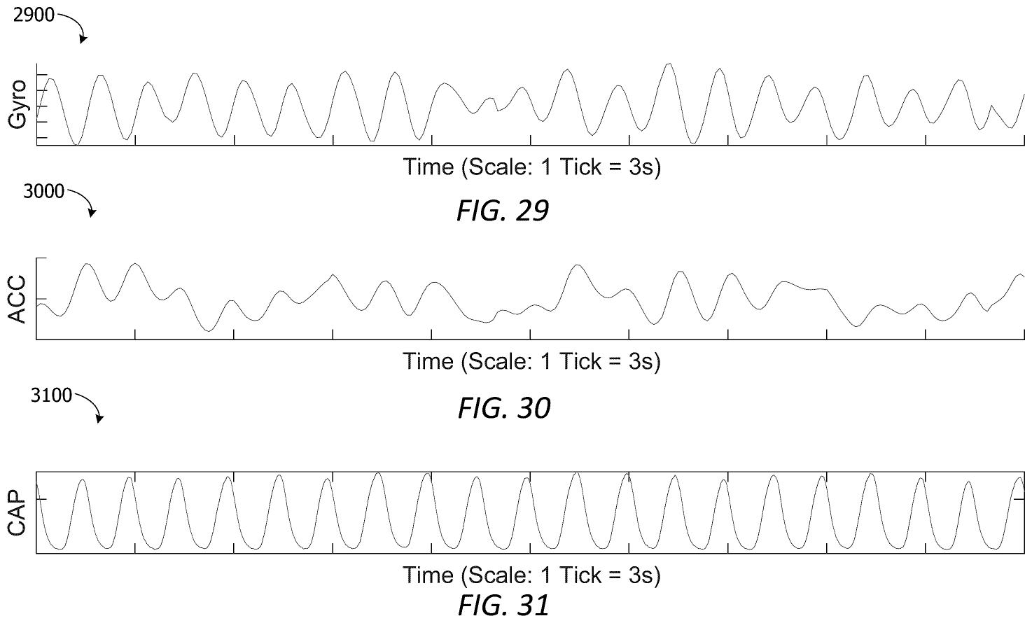

FIG. 29, FIG. 30, and FIG. 31 show example respiration waveforms from a participant at high RR of 40 brpm. FIG. 29 is an exemplary line graph 2900 illustrating a respiration waveform derived from gyroscope (GYRO) sensor. The horizontal axis (x-axis) shows change over time, where one tick equals three seconds (3 s). FIG. 30 is an exemplary line graph 3000 illustrating respiration waveform derived from ACC signal. The horizontal axis (x-axis) shows change over time, where one tick equals three seconds (3 s). FIG. 31 is an exemplary line graph 3100 illustrating a reference end-tidal CO₂ waveforms from a standard capnograph (CAP) with RR at 40 brpm. The horizontal axis (x-axis) shows change over time, where one tick equals three seconds (3 s).

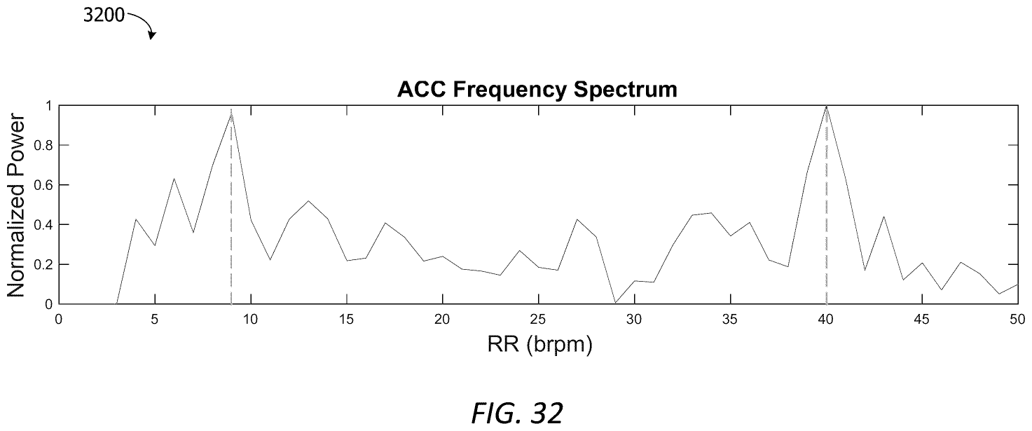

FIG. 32 is an exemplary line graph 3200 illustrating a spectrum of ACC respiration signal with a false frequency peak near 9 brpm alongside a true respiratory frequency peak at 40 brpm. The graph 3200 includes a vertical axis (y-axis) showing normalized power. The horizontal axis (x-axis) shows change in respiratory rate (RR) in breaths-per-minute (brpm). Gyro waveform has more pronounced oscillations without baseline wander and other artifacts compared to ACC, as shown in FIG. 29 above. Spectrum of ACC respiration signal with baseline wander showing a peak near 9 brpm can suppress true estimate if noise increases. A higher weightage to Gyro RR can result in more reliable RR estimate.

Exemplary fusion equations, in some examples, include both expert rule-based selection of estimates and trained methods, such as regression models. One example of a rule-based selection, which incorporates information about what RR ranges, motion, and postures that the various estimates perform best at, is shown below:

$$\{ \begin{matrix}{RR}_{F} = {RR}_{G} & \begin{matrix}if MV = ' Rest ' AND Po = \\ ' Upright ' AND RRQ > 50\end{matrix} \\ {RR}_{F} = {RR}_{A} & \begin{matrix}elseif MV = ' Rest ' AND Po \neq \\ \begin{matrix}' Upright ' AND {RR}_{F}^{i - 1} < \\ 12 AND {RRQ}_{A} > 50\end{matrix}\end{matrix} \\ {RR}_{F} = {RR}_{G} & \begin{matrix}elseif MV = ' Rest ' AND Po \neq \\ \begin{matrix}' Upright ' AND {RR}_{F}^{i - 1} > \\ 20 AND {RRQ}_{G} > 50\end{matrix}\end{matrix} \\ {RR}_{F} = {RR}_{P} & elseif MV \neq ' Rest ' AND {RRQ}_{P} > 80 \\ {RR}_{F} = Invalid & \begin{matrix}elseif {RRQ}_{P} < 50 AND {RRQ}_{A} < \\ 50 AND {RRQ}_{G} < 50\end{matrix} \\ {RR}_{F} = Quality Weighted Average & otherwise\end{matrix}$$

Here, RR_{F} is the final estimate of RR, while RR_{G}, RR_{A}, and RR_{P} are the gyroscope, accelerometer, and photoplethysmogram-based estimates of RR respectively, for which RRQ are the respective quality metrics. MV is the movement level assessed from the accelerometer MA data and Po is the posture from ACC and gyroscope.

An example of a regression based fusion model is shown as follows:

$$R {R}_{F} = \frac{{w}_{A} * R {R}_{A} + {w}_{G} * R {R}_{G} + {w}_{P} * R {R}_{P}}{{w}_{A} + {w}_{G} + {w}_{P}}$$

$$where$$

$${w}_{X} = {C}_{X} * R R {Q}_{X} * ( \frac{1}{abs ( {T}_{X} - R {R}_{X} )} )$$

In this approach, the weighted average of the three estimates is taken with a weight wₓ, for each estimate. The weights are computed by learning scaling coefficients Cₓ and best target respiration rate Tₓ for each of the three modalities based on existing data. In this way, the estimate is weighted more heavily when it is near the target RR value for that estimate. Additionally, these values can be trained separately for different conditions such as postures and activities, thus giving more weight to some modes in some conditions such as emphasizing gyroscope more heavily when in the upright posture.

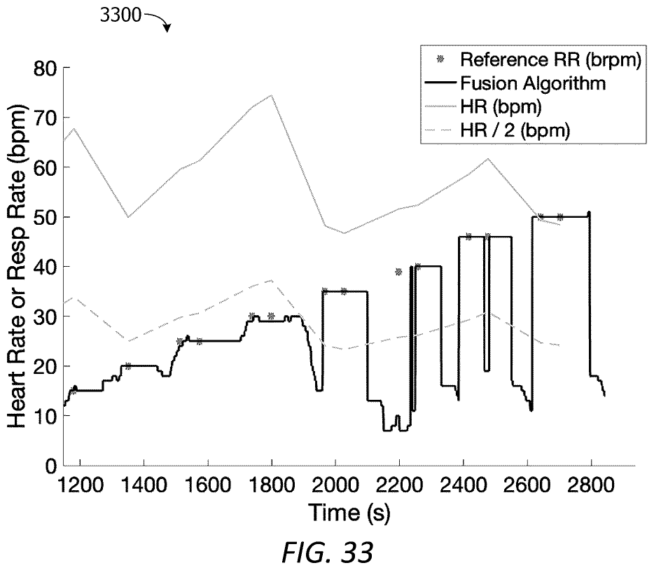

FIG. 33 is an exemplary line graph 3300 illustrating final RR outputs during a sequence of paced breathing tests. The line graph 3300 shows a sample of final respiration rate outputs of the estimation methods during a paced breathing protocol.

Example output of the respiration rate estimation method during a paced breathing protocol is presented in FIG. 33 along with the reference RR (measured by EtCO₂ capnography) over the ranges from 5-50 brpm in steps of 5 brpm, and simultaneously measured HR values. The RR estimation method faithfully tracks the true RR up to and including 50 breaths per minute. Standard methods of RR estimation from PPG alone are not able to predict rates above half of the heart rate due to a Nyquist frequency limitation. However, the proposed method has no difficulty with the higher respiration rates.

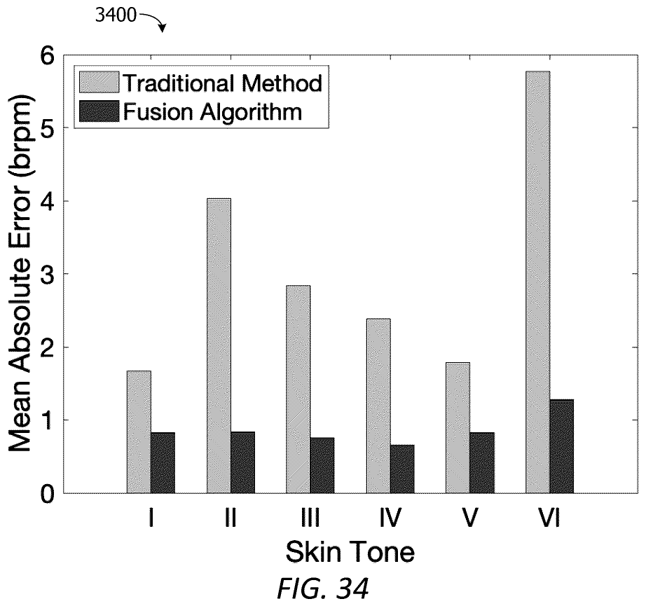

FIG. 34 is an exemplary bar graph 3400 illustrating comparison of RR mean absolute error performances among skin tones groups of I-VI for a traditional literature model and the RR manager implemented fusion model. The standard model performs significantly worse for some skin tones than others, particularly for the darkest skin tone VI, whereas the RR manager implemeting the fusion method described herein shows little to no difference.

Another strength of the proposed invention combining both the PPG and ACC sensing methods is highlighted in FIG. 34, which exhibits the error rates between the darker and lighter skin tones. Current (standard) models that rely solely on PPG show relatively poor error rates overall, but significantly higher error rates for the darkest skin tones due to greater light absorption resulting in weaker signal-to-noise ratio. In contrast, the proposed RR method is insensitive to darker skin tones resulting in high accuracies or lower error rates for all skin tones tested.

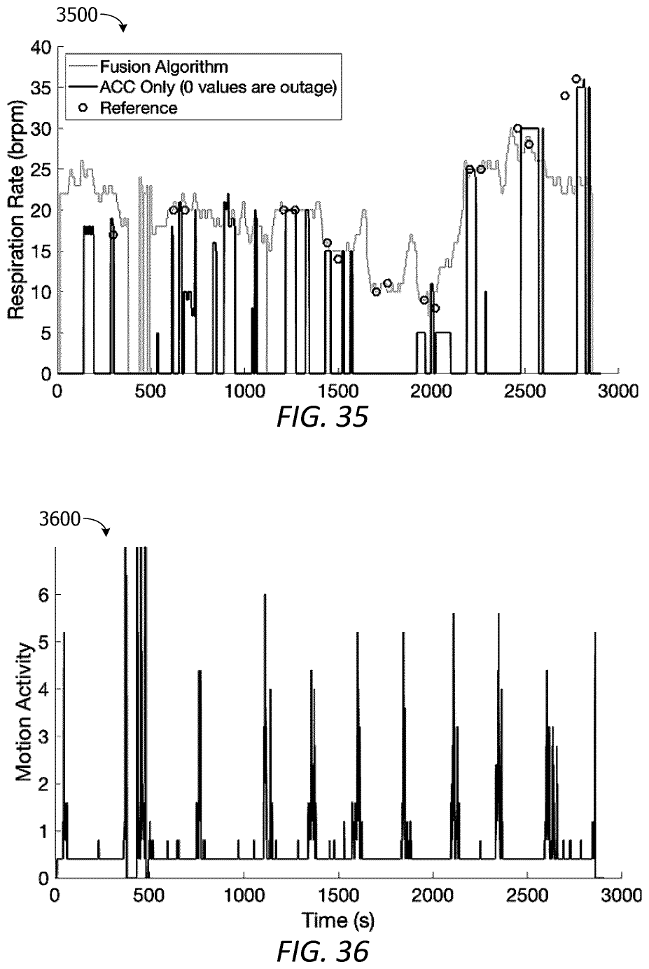

FIG. 35 is an exemplary line graph 3500 illustrating example recording of a subject that illustrates reliable performance of the RR manager fusion method compared to an ACC-based subset during high motion activity. FIG. 36 is an exemplary line graph 3600 illustrating motion activity variation over time for a subject with high activity correlated to less reliable ACC respiration rate estimate in FIG. 35. RR cannot be estimated from ACC (or Gyro) alone in some motion conditions, but the PPG mode estimation is able to compensate when used in combination. Thus, FIG. 36 shows motion activity variation over time for a subject while FIG. 35 shows less reliable ACC estimate during high motion.

In parallel, the PPG respiratory rate estimate can compensate for periods of time when the ACC estimate is less reliable, such as during higher motion activity such as the recording presented in FIG. 35 and FIG. 36. Although ACC alone can produce direct, accurate RR estimations during perfectly still conditions, it can suffer from low coverage in real-world applications with greater movement. Both PPG and ACC mode estimations complement each other to track RR accurately and continuously throughout this and other recordings.

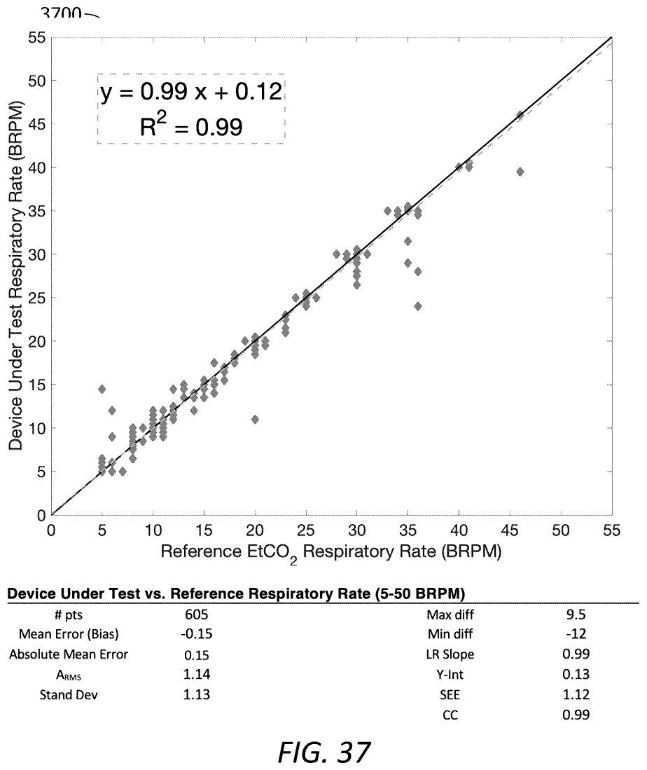

FIG. 37 is an exemplary correlation scatter graph 3700 illustrating clinical validation results for wearable armband devices embedded with the RR manager implemented with the proposed RR methods tested on 20 subjects, in one example. Clinical validation results of the proposed RR method implemented on a wearable device for real-time prospective evaluation of RR performance are shown in FIG. 37. The root mean square error rates are shown to be very low ˜1 brpm with a very high correlation of 0.99 across a wide range of reference RR values between five brpm and fifty brpm (5-50 brpm). In some examples, the range of interest is a limited range between five brpm and thirty-five brpm (5-35 brpm).

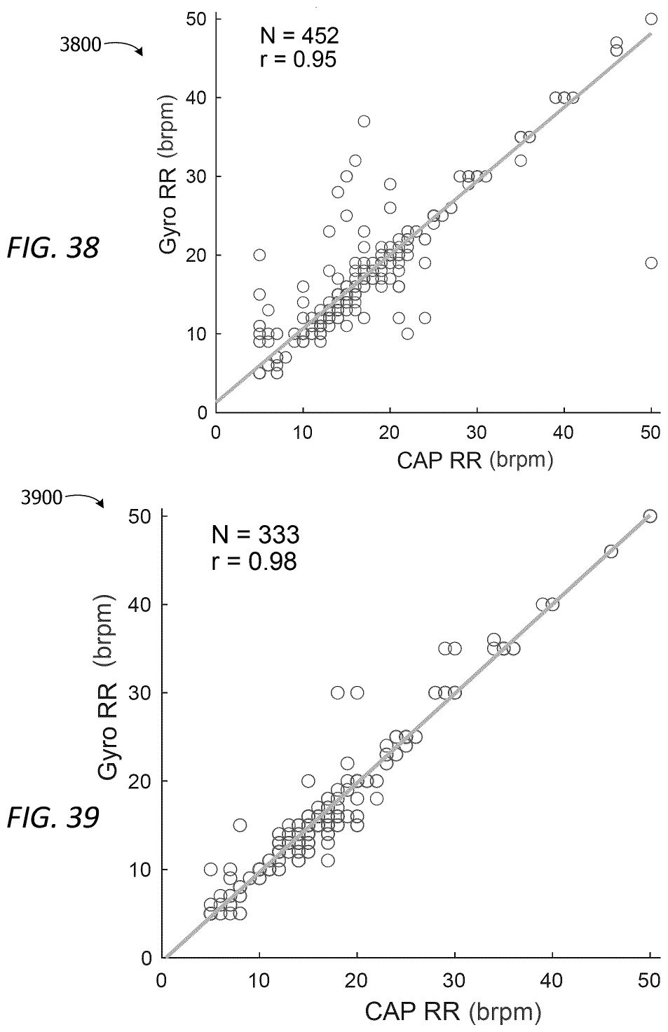

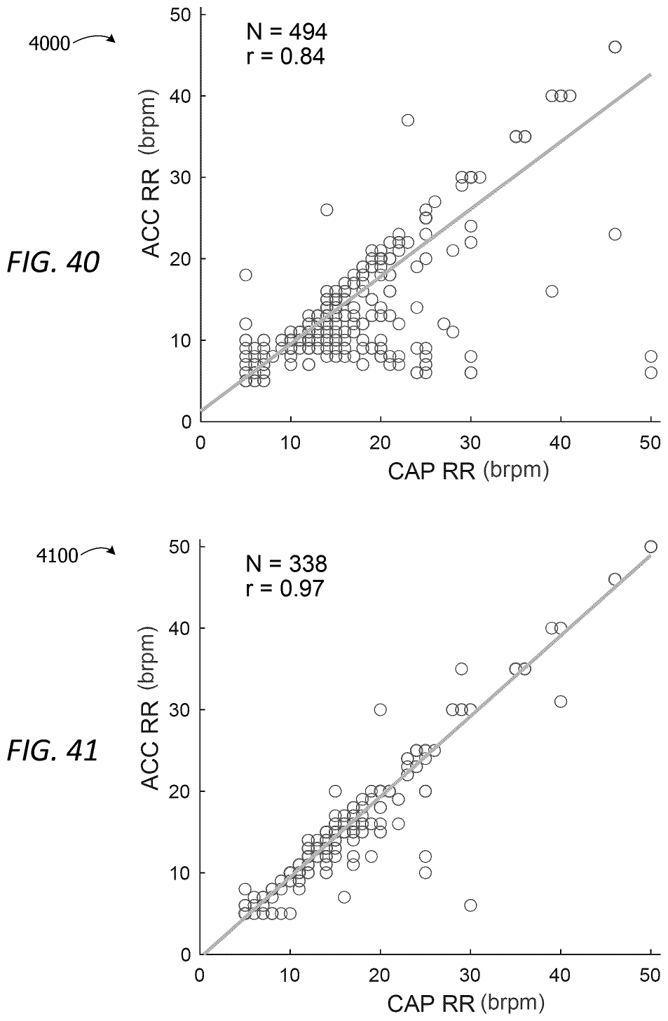

FIG. 38 is an exemplary line graph 3800 illustrating a plot showing correlation of the gyroscope RR estimate with the reference CAP RR where the user is in an upright position. FIG. 39 is an exemplary line graph 3900 illustrating a correlation between the reference CAP RR and the RR generated using gyroscope sensor data while the user is in a supine position. FIG. 40 is an exemplary line graph 4000 illustrating correlation between the reference CAP RR and the ACC-based RR estimate where the user is in an upright position. FIG. 41 is an exemplary line graph 4100 illustrating correlation between the reference CAP RR and the ACC-based RR estimate where the user is in the supine position.

FIG. 38, FIG. 39, FIG. 40, and FIG. 41 show correlation of reference CAP RR estimate with gyroscope (FIG. 38 and FIG. 39) and ACC (FIG. 40 and FIG. 41) in upright and supine postures, tested on 30 subjects. ACC has relatively reduced correlation and increased error in upright, particularly in standing posture. Each scatter point represents 1-min RR estimate in brpm, with count indicated by N and correlation with r; solid line is the least-square fit line.

FIG. 38, FIG. 39, FIG. 40, and FIG. 41 show correlation plots of gyroscope and ACC respiration rate with reference respiration rate by Capnography (CAP RR) in upright (sitting or standing) and supine postures. The gyroscope shows higher correlation (r) and lower mean absolute error (MAE) than ACC, particularly in upright posture, MAE_{upright} (CAP, Gyro)=1.10 and MAE_{upright} (CAP, ACC)=2.3 brpm. A higher weightage to gyroscope estimate will result in improved IMU sensor performance.

Further, superior performance of gyroscope-based respiration rate calculation has been observed in normal (12-20 brpm) and high RR range (>20 brpm), with MAE_{RR-High}(CAP, Gyro)=0.6 brpm; MAE_{RR-High}(CAP, ACC)=3.2 brpm in high RR range. In low RR range (<12 brpm), however, ACC outperforms with MAE_{RR-Low}(CAP, ACC)=0.8 brpm, MAE_{RR-Low} (CAP, Gyro)=1.6 brpm before harmonic correction and MAE_{RR-Low} (CAP, Gyro)=0.9 brpm after harmonic correction using a threshold T=0.65. For RR>20 brpm, harmonic correction slightly worsens the performance, which agrees with the observation that harmonic issues exist mainly at low RR and the correction should be restricted for maximum peak detected between 10-20 brpm.

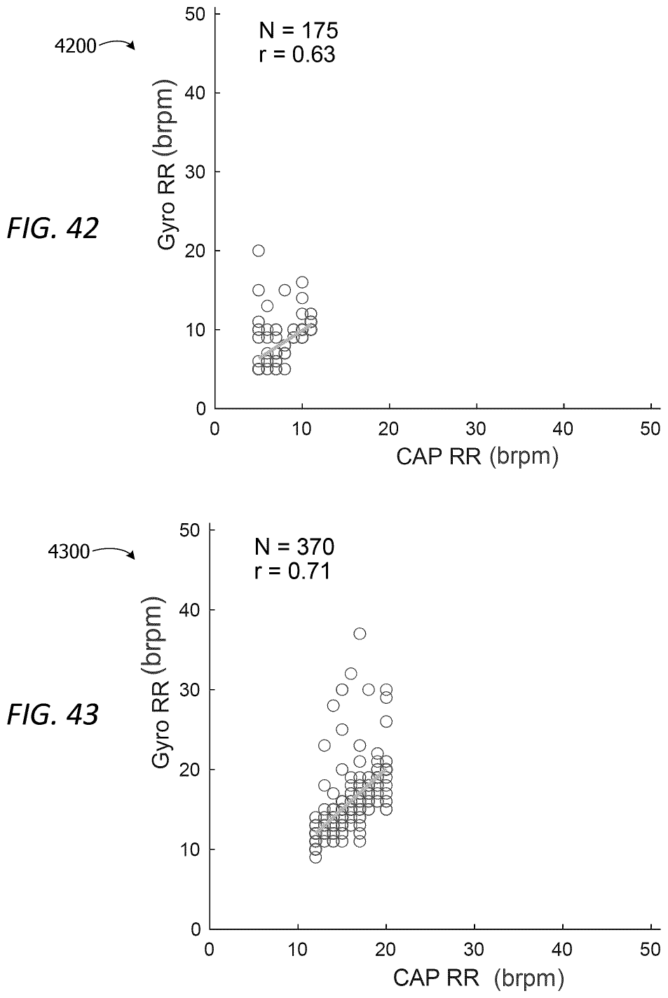

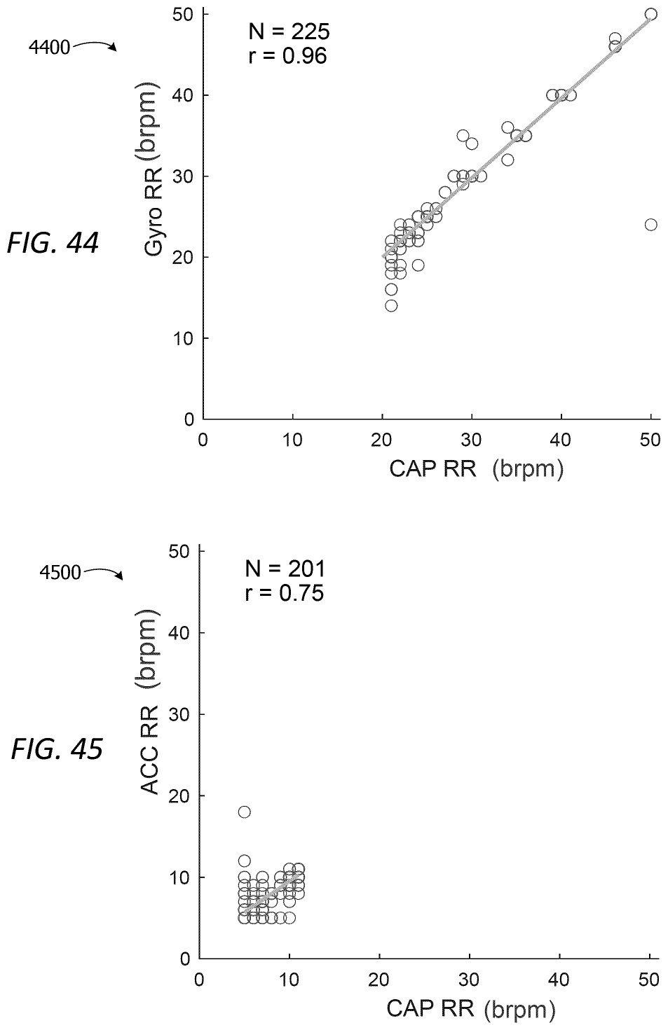

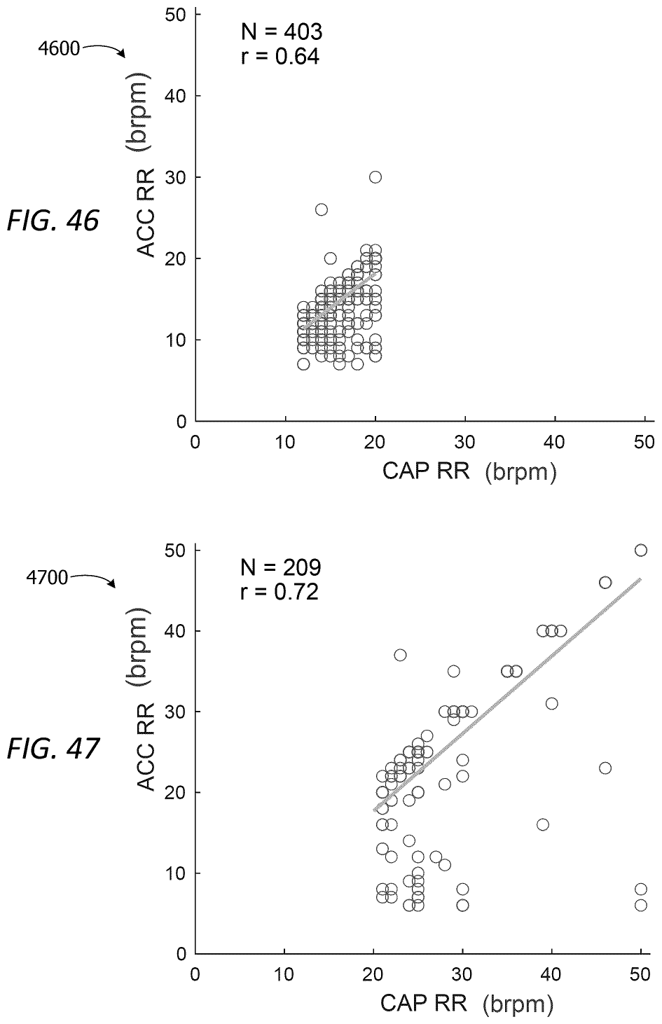

FIG. 42 is an exemplary line graph 4200 illustrating a plot showing correlation of reference CAP RR estimate with gyroscope RR estimate in a low RR range. FIG. 43 is an exemplary line graph 4300 illustrating a plot showing correlation of reference CAP RR estimate with gyroscope RR estimate in a normal RR range. FIG. 44 is an exemplary line graph 4400 illustrating a plot showing correlation of reference CAP RR estimate with gyroscope RR estimate in a high RR range. FIG. 45 is an exemplary line graph 4500 illustrating a plot showing correlation of reference CAP RR estimate with ACC RR estimate in a low RR range. FIG. 46 is an exemplary line graph 4600 illustrating a plot showing correlation of reference CAP RR estimate with ACC RR estimate in a normal RR range. FIG. 47 is an exemplary line graph 4700 illustrating a plot showing correlation of reference CAP RR estimate with ACC RR estimate in a high RR range.

FIGS. 42-47 show correlation plots of gyroscope and ACC RR estimates with ground truth CAP RR at different RR ranges, showing above observations. Thus, accelerometer and gyroscope RR fusion can benefit from weightage impacted by RR range, determined by previous or current RR estimates.

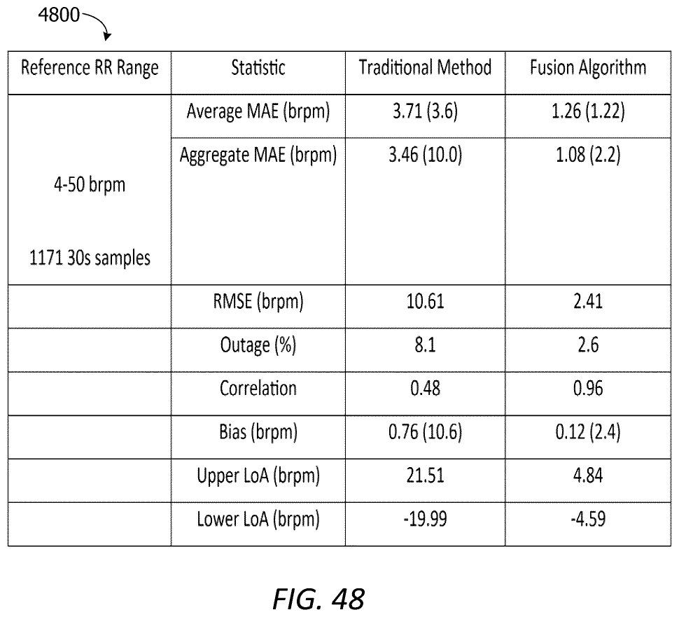

FIG. 48 is an exemplary table 4800 illustrating comparison of the RR manager algorithm with a traditional literature algorithm. Numbers in parentheses indicate one (1) standard deviation across subjects (Average MAE) or across samples (Aggregate MAE and Bias).

Comparison of a proposed RR estimation method with a traditional literature algorithm (PPG Smart Fusion) is detailed in table 4800. The dataset is comprised of both spontaneous breathing and paced (metronome) breathing. The proposed method shows an error rate substantially lower than the literature method, with correspondingly very low bias and high correlation to the reference RR measured by the standard (traditional) capnograph. The method shows strong performance for the full RR range up to 50 brpm.

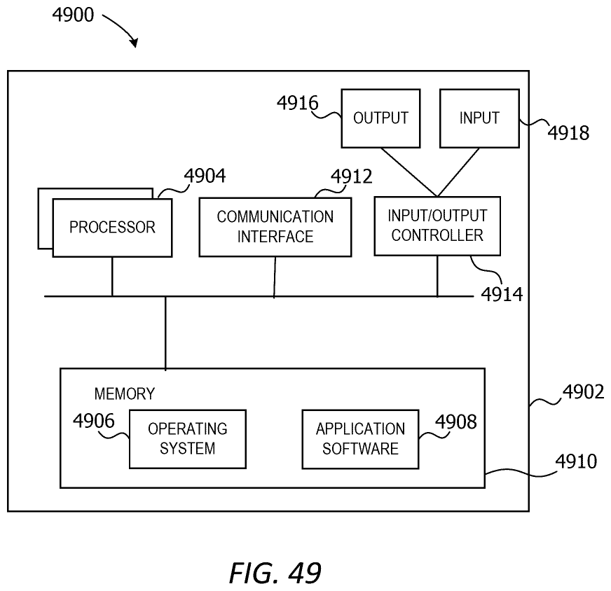

Turning now to FIG. 49, a computing apparatus 4902 is shown. The present disclosure is operable with a computing system 4900 including computing device, such as, but not limited to, the computing apparatus 4902. The computing apparatus is a computing device, such as, but not limited to, the computing device 102 in FIG. 1. In an embodiment, components of the computing apparatus 4902 may be implemented as a part of an electronic device according to one or more embodiments described in this specification.

The computing apparatus 4902 comprises one or more processors 4904 which may be microprocessors, controllers, or any other suitable type of processors for processing computer executable instructions to control the operation of the electronic device. The one or more processors include a processing device, such as, but not limited to, the processor 106 in FIG. 1. Alternatively, or in addition, the processor 4904 is any technology capable of executing logic or instructions, such as a hardcoded machine.

Platform software comprising an operating system 4906 or any other suitable platform software may be provided on the apparatus 4902 to enable application software 4908 to be executed on the device.

Computer executable instructions may be provided using any computer-readable media that are accessible by the computing apparatus 4902. Computer-readable media may include, for example, computer storage media such as a memory 4910 and communications media. The memory 4910 may be any type of memory or computer storage media, such as, but not limited to, the memory 108 in FIG. 1.

Computer storage media, such as a memory 4910, include volatile and non-volatile, removable, and non-removable media implemented in any method or technology for storage of information such as computer readable instructions, data structures, program modules or the like. Computer storage media include, but are not limited to, RAM, ROM, EPROM, EEPROM, persistent memory, phase change memory, flash memory or other memory technology, CD-ROM, digital versatile disks (DVD) or other optical storage, magnetic cassettes, magnetic tape, magnetic disk storage, shingled disk storage or other magnetic storage devices, or any other non-transmission medium that can be used to store information for access by a computing apparatus.

In contrast, communication media may embody computer readable instructions, data structures, program modules, or the like in a modulated data signal, such as a carrier wave, or other transport mechanism. As defined herein, computer storage media do not include communication media. Therefore, a computer storage medium should not be interpreted to be a propagating signal per se. Propagated signals per se are not examples of computer storage media. Although the computer storage medium (the memory 4910) is shown within the computing apparatus 4902, it will be appreciated by a person skilled in the art, that the storage may be distributed or located remotely and accessed via a network or other communication link (e.g., using a communication interface 4912).

The computing apparatus 4902 may comprise an input/output controller 4914 configured to output information to one or more output devices 4916, for example a display or a speaker, which may be separate from or integral to the electronic device. The input/output controller 4914 may also be configured to receive and process an input from one or more input devices 4918, for example, a keyboard, a microphone, or a touchpad. In one embodiment, the output device 4916 may also act as the input device. An example of such a device may be a touch sensitive display. The input/output controller 4914 may also output data to devices other than the output device, e.g., a locally connected printing device. In some embodiments, a user may provide input to the input device(s) 4918 and/or receive output from the output device(s) 4916.

The functionality described herein can be performed, at least in part, by one or more hardware logic components. According to an embodiment, the computing apparatus 4902 is configured by the program code when executed by the processor 4904 to execute the embodiments of the operations and functionality described. Alternatively, or in addition, the functionality described herein can be performed, at least in part, by one or more hardware logic components. For example, and without limitation, illustrative types of hardware logic components that can be used include Field-programmable Gate Arrays (FPGAs), Application-specific Integrated Circuits (ASICs), Program-specific Standard Products (ASSPs), System-on-a-chip systems (SOCs), Complex Programmable Logic Devices (CPLDs), Graphics Processing Units (GPUs).

At least a portion of the functionality of the various elements in the figures may be performed by other elements in the figures, or an entity (e.g., processor, web service, server, application program, computing device, etc.) not shown in the figures.

### Additional Examples

In some examples, for PPG processing, the system applies an adaptive filter based on previous RR and modality (e.g., filter for Mayer waves specifically for amplitude modulation) rather than a fixed one. The system identifies individual breath cycles derived from PPG, and determines their reliability on an individual basis to select only cycles the system is confident enough in to include. For combining ACC or Gyro axes, the system uses PCA or other combination methods to determine the axis in which the respiratory movement occurs and isolate only the respiratory movements in that direction. The combination of axes from ACC and/or gyroscope can be used to determine posture, motion activity and/or movement levels. The system uses such additional information to help determine the final RR selection.

In other examples, the system uses information on recent high quality RR estimates to track RR through more difficult real-world conditions. The RR estimate is not merely based on comparisons from current sensor readings only. Instead, the system considers the previous history as well.

The system, in still other examples, uses motion derived from IMU to impact processing of IMU and PPG data, where high motion leads to stricter band-pass filtering in PPG and rejection of high motion partial segment in IMU. The system further identifies and corrects respiration harmonics present in Gyro signal at low respiratory rates, which improves the RR estimate from IMU sensor. Further, ACC and Gyro comparison and unique selection based on posture, RR range and quality improves accuracy and reliability of RR monitoring. In some examples, the respiratory rate is estimated based on PPG, ACC, and gyroscope data.

The PPG sensor processing uses PPG signals at any wavelengths of green, red and/or infrared (IR). Amplitude, baseline (intensity) and frequency variation waveforms are estimated and separately processed. The PPG sensor processing includes refined adaptive filter based on previous RR estimate if high motion is present. The respiratory surrogate waveforms derived from PPG such as amplitude modulation has different filter(s) to reduce Mayer wave impact based on previous RR. The system further utilizes a set of rules to isolate reliable breaths that includes motion. The system further includes a quality metric based on coefficient of variation among inter breath intervals or amplitudes used to estimate the quality of the RR output(s).

In still other examples, the system includes movement sensor processing which uses PCA to combine multiple axes of ACC or gyroscope data. It performs tracking and tracing of respiratory frequency (TTRF) using the time frequency spectra (TFS) such as short-time Fourier Transform (STFT). The quality estimate is clearly explained using latent of first principal component (PC) or kurtosis. The system selects one if only one of ACC or gyroscope is of high quality. Otherwise, the system uses posture data and RR range (from previous RR estimate) criteria to select ACC vs gyroscope estimate. Otherwise, the system uses an average (e.g., weighted average).

The system includes activity considerations in which RR estimation utilizes motion activity (MA) less than threshold near a respiratory interval estimation. For ACC, partial segment is dropped before PCA analysis or invalidated if entire segment has high act. MA level estimation is also generated based on an equation.

In some examples, the system provides an algorithm for PPG and movement integration. If output of IMU and IMU derived estimate has higher quality, it is used, otherwise PPG is used and reported, if PPG is of good quality.

In still other examples, the system provides high quality-based selection using posture data and RR range. Multiple ACC data or data from multiple gyroscope sensors may be utilized.

For PPG processing, an adaptive filter based on previous RR and modality (e.g., filter for Mayer waves specifically for amplitude modulation) rather than a fixed one is utilized. The system additionally identifies individual breath cycles derived from PPG, and the system determines their reliability on an individual basis to select only cycles having sufficiently high confidence and reliability.

In some examples, for combining across ACC or Gyro axes, the system uses PCA or other combination methods to determine the axis in which the respiratory movement occurs and isolate only the respiratory related movements in that direction. The combination of ACC and/or Gyro axes can be used to determine posture, motion activity and movement levels. Posture states such as supine, upright, or standing, motion/movement levels, and the previous range of RR estimate are taken into account for the selection of respiratory modes and final RR. The system uses posture, motion, RR range information to help determine the final RR selection.

In other examples, the system uses information on recent high quality RR estimates to track RR through more difficult real-world conditions. The system uses motion derived from IMU to impact processing of IMU and PPG data, where high motion leads to stricter band-pass filtering in PPG and rejection of high motion partial segment in IMU. The system also identifies and corrects respiration harmonics present in Gyro signal at lower respiratory rates, which improves the RR estimate from IMU sensor.

Alternatively, or in addition to the other examples described herein, examples include any combination of the following:
wherein the set of rules further comprises an adaptive peak-to-trough threshold, wherein an amplitude of a PPG signal greater than the threshold is acceptable, and wherein the PPG signal is disregarded as noise where the amplitude of the PPG signal is less than the threshold;
wherein the peak-to-trough threshold is adaptively updated based on a percentage of previous breaths occurring within a predetermined time-period;
a motion activity threshold, wherein motion activity around an interval associated with an IMU-based respiratory signal less than a predetermined threshold value is used for calculating an estimated respiratory rate, and wherein motion activity around the interval greater than the predetermined threshold is filtered out;
a breath duration rule, wherein a breath duration is required to be within range of desired duration compared to predetermined set of previous breath intervals;
select the PPG-based respiratory rate as the final respiratory rate where the quality metric for the IMU-based respiratory rate indicating the IMU-based respiratory rate is less reliable than the PPG-based respiratory rate;
select the IMU-based respiratory rate as the final respiratory rate where the quality metric for the IMU-based respiratory rate indicating the IMU-based respiratory rate is more reliable than the PPG-based respiratory rate;
update the set of rules by a machine learning model to adaptively improve accuracy of respiratory rate calculation;
receiving PPG-based sensor data from a PPG sensor device and IMU-based sensor data from an IMU sensor device;
applying a set of rules for filtering and processing the sensor data, wherein the set of rules comprises at least one rule for isolating breath-related peaks in PPG signals from the PPG sensor data and identifying motion activity associated with respiration from IMU sensor data;
generating respiratory rate estimates, including a PPG-based respiratory rate associated with the PPG sensor data and an IMU-based respiratory rate estimate based on IMU sensor data in parallel;
selecting a final respiratory rate from the plurality of respiratory rate estimates based on a quality metric, wherein the quality metric comprises a quality score for each respiratory rate estimate indicating reliability of a given respiratory rate estimate;
a quality estimate, including a quality score or other value indicating quality of the calculated respiratory rate;
providing the final respiratory rate and quality score to a user via a user interface, wherein the respiratory rate is a continuous respiration rate of a user wearing the set of sensor devices;
wherein the IMU-based respiratory rate estimate comprises at least one of accelerometer-based respiratory rate estimate generated using sensor data from an accelerometer and gyroscope-based respiratory rate estimate generated based on sensor data generated by a gyroscope; and
updating at least one threshold value associated with the set of rules by a machine learning model in real-time.

In some examples, the operations illustrated in FIGS. 9-13 can be implemented as software instructions encoded on a computer-readable medium, in hardware programmed or designed to perform the operations, or both. For example, aspects of the disclosure can be implemented as a system on a chip or other circuitry including a plurality of interconnected, electrically conductive elements.

While the aspects of the disclosure have been described in terms of various examples with their associated operations, a person skilled in the art would appreciate that a combination of operations from any number of different examples is also within scope of the aspects of the disclosure.

The term “Wi-Fi” as used herein refers, in some examples, to a wireless local area network using high frequency radio signals for the transmission of data. The term “BLUETOOTH®” as used herein refers, in some examples, to a wireless technology standard for exchanging data over short distances using short wavelength radio transmission. The term “NFC” as used herein refers, in some examples, to a short-range high frequency wireless communication technology for the exchange of data over short distances.

### Exemplary Operating Environment

Exemplary computer-readable media include flash memory drives, digital versatile discs (DVDs), compact discs (CDs), floppy disks, and tape cassettes. By way of example and not limitation, computer-readable media comprise computer storage media and communication media. Computer storage media include volatile and nonvolatile, removable, and non-removable media implemented in any method or technology for storage of information such as computer-readable instructions, data structures, program modules and the like. Computer storage media are tangible and mutually exclusive to communication media. Computer storage media are implemented in hardware and exclude carrier waves and propagated signals. Computer storage media for purposes of this disclosure are not signals per se. Exemplary computer storage media include hard disks, flash drives, and other solid-state memory. In contrast, communication media typically embody computer-readable instructions, data structures, program modules, or the like, in a modulated data signal such as a carrier wave or other transport mechanism and include any information delivery media.

Although described in connection with an exemplary computing system environment, examples of the disclosure are capable of implementation with numerous other special purpose computing system environments, configurations, or devices.

Examples of well-known computing systems, environments, and/or configurations that can be suitable for use with aspects of the disclosure include, but are not limited to, mobile computing devices, personal computers, server computers, hand-held or laptop devices, multiprocessor systems, gaming consoles, microprocessor-based systems, set top boxes, programmable consumer electronics, mobile telephones, mobile computing and/or communication devices in wearable or accessory form factors (e.g., watches, glasses, headsets, or earphones), network PCs, minicomputers, mainframe computers, distributed computing environments that include any of the above systems or devices, and the like. Such systems or devices can accept input from the user in any way, including from input devices such as a keyboard or pointing device, via gesture input, proximity input (such as by hovering), and/or via voice input.

Examples of the disclosure can be described in the general context of computer-executable instructions, such as program modules, executed by one or more computers or other devices in software, firmware, hardware, or a combination thereof. The computer-executable instructions can be organized into one or more computer-executable components or modules. Generally, program modules include, but are not limited to, routines, programs, objects, components, and data structures that perform tasks or implement abstract data types. Aspects of the disclosure can be implemented with any number and organization of such components or modules. For example, aspects of the disclosure are not limited to the specific computer-executable instructions, or the specific components or modules illustrated in the figures and described herein. Other examples of the disclosure can include different computer-executable instructions or components having more functionality or less functionality than illustrated and described herein.

In examples involving a general-purpose computer, aspects of the disclosure transform the general-purpose computer into a special-purpose computing device when configured to execute the instructions described herein.

The examples illustrated and described herein as well as examples not specifically described herein but within the scope of aspects of the disclosure constitute exemplary means for continuous respiratory rate monitoring using a wearable sensor device. For example, the elements illustrated in FIGS. 1-8 such as when encoded to perform the operations illustrated in FIGS. 9-13, constitute exemplary means for receiving PPG-based sensor data from a PPG sensor device and IMU-based sensor data from an IMU sensor device; exemplary means for applying a set of rules for filtering and processing the sensor data, wherein the set of rules comprises at least one rule for isolating breath-related peaks in PPG signals from the PPG sensor data and identifying body motion or movement associated with respiration from IMU sensor data; exemplary means for generating respiratory rate estimates, including a PPG-based respiratory rate associated with the PPG sensor data and an IMU-based respiratory rate estimate based on IMU sensor data in parallel; and exemplary means for selecting a final respiratory rate from the plurality of respiratory rate estimates based on a quality metric, wherein the quality metric comprises a quality score for each respiratory rate estimate indicating reliability of a given respiratory rate estimate; and exemplary means for providing the final respiratory rate and quality score to a user via a user interface, wherein the respiratory rate is a continuous respiration rate of a user wearing the set of sensor devices.

The order of execution or performance of the operations in examples of the disclosure illustrated and described herein is not essential, unless otherwise specified. That is, the operations can be performed in any order, unless otherwise specified, and examples of the disclosure can include additional or fewer operations than those disclosed herein. For example, it is contemplated that executing or performing an operation before, contemporaneously with, or after another operation is within the scope of aspects of the disclosure.

The indefinite articles “a” and “an,” as used in the specification and in the claims, unless clearly indicated to the contrary, should be understood to mean “at least one.” The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used shall only be interpreted as indicating exclusive alternatives (i.e., “one or the other but not both”) when preceded by terms of exclusivity, such as “either” “one of” “only one of” or “exactly one of.” “Consisting essentially of,” when used in the claims, shall have its ordinary meaning as used in the field of patent law.

As used in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The use of “including,” “comprising,” “having,” “containing,” “involving,” and variations thereof, is meant to encompass the items listed thereafter and additional items.

Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense.

What is claimed is:

1. A system for respiratory rate monitoring, the system comprising:
a processor;
a set of sensor devices generating sensor data, the set of sensor devices comprising a photoplethysmography (PPG) sensor and an inertial movement unit (IMU) sensor, the sensor data comprising PPG sensor data and IMU sensor data; and
a computer-readable medium storing instructions that are operative upon execution by the processor to:
generate, by a respiratory rate estimator, a plurality of respiratory rate estimates from the sensor data, the plurality of respiratory rate estimates comprising PPG-based respiratory rate estimates based on wavelengths of PPG signals extracted from the PPG sensor data and IMU-based respiratory rate estimates based on accelerometer and gyroscope sensor data in parallel;
combine, by a respiratory rate fusion model, the plurality of respiratory rate estimates into a final PPG-based respiratory rate estimate and a final IMU-based respiratory rate estimate;
select, by a selection manager, a final respiratory rate from the plurality of respiratory rate estimates based on a set of performance optimization models and a set of rules maintained by a rules engine, the plurality of respiratory rate estimates comprising a final PPG-based respiratory rate estimate and a final IMU-based respiratory rate estimate; and
output the final respiratory rate and a quality estimate associated with the final respiratory rate via a user interface, wherein the respiratory rate is a continuous respiration rate of a user wearing the set of sensor devices.

2. The system of claim 1, wherein the set of rules further comprises an adaptive peak-to-trough threshold, wherein an amplitude of a PPG signal greater than the peak-to-trough threshold is acceptable, and wherein the PPG signal is disregarded as noise where the amplitude of the PPG signal is less than the peak-to-trough threshold.

3. The system of claim 1, further comprising:
perform signal processing to filter the sensor data, by the respiratory rate estimator, wherein the signal processing includes filtering to remove Mayer-wave in-band noise, transients, and motion artifacts.

4. The system of claim 1, wherein the instructions are further operative to:
perform signal processing on IMU-based respiratory signals using time-frequency spectrum (TFS) based tracking and adaptive selection of IMU signal modes based on movement, posture, activity, respiratory rate range, and time-frequency spectral features.

5. The system of claim 1, wherein the set of rules further comprises:
a breath duration rule, wherein a breath duration is required to be within range of desired duration compared to predetermined set of previous breath intervals;
tracking and tracing of respiratory frequency using current and previous respiratory rate estimates for accelerometer and gyroscope fusion; and
harmonic correction of gyroscope respiratory rate estimate when current PPG or ACC or previous respiratory rate estimates are in low range.

6. The system of claim 1, wherein the instructions are further operative to:
select the PPG-based respiratory rate estimate as the final respiratory rate where a quality metric for the IMU-based respiratory rate estimate indicates the IMU-based respiratory rate estimate is less reliable than the PPG-based respiratory rate estimate; and
select the IMU-based respiratory rate estimate as the final respiratory rate where the quality metric for the IMU-based respiratory rate estimate is more reliable than the PPG-based respiratory rate estimate.

7. The system of claim 1, wherein the instructions are further operative to:
update the set of rules by a respiratory rate performance optimization machine learning model to improve accuracy of respiratory rate calculation.

8. A computer-implemented method for respiratory rate monitoring, the computer-implemented method comprising:
receiving photoplethysmography (PPG) sensor data from a PPG sensor device and inertial movement unit (IMU) sensor data from an IMU sensor device;
applying a set of rules for filtering and processing the sensor data, wherein the set of rules comprises at least one rule for isolating breath-related peaks in PPG signals from the PPG sensor data and identifying motion activity associated with respiration from the IMU sensor data;
generating respiratory rate estimates, including a PPG-based respiratory rate estimate based on the PPG sensor data and an IMU-based respiratory rate estimate based on the IMU sensor data in parallel;
selecting a final respiratory rate from the respiratory rate estimates based on a quality metric, wherein the quality metric comprises a quality score for each respiratory rate estimate indicating reliability of a given respiratory rate estimate; and
identifying the final respiratory rate and the quality score.

9. The computer-implemented method of claim 8, wherein applying the set of rules further comprises:
filtering out a PPG signal having an amplitude less than an adaptive peak-to-trough threshold.

10. The computer-implemented method of claim 8, further comprising:
applying a motion activity threshold, wherein motion activity around an interval associated with an IMU-based respiratory signal less than a predetermined threshold value is used for calculating an estimated respiratory rate, and wherein motion activity around the interval greater than the predetermined threshold value is filtered out.

11. The computer-implemented method of claim 8, further comprising:
applying a breath duration rule, wherein a breath duration is required to be within range of desired duration compared to predetermined set of previous breath intervals.

12. The computer-implemented method of claim 8, further comprising:
selecting the PPG-based respiratory rate as the final respiratory rate where the quality metric for the IMU-based respiratory rate indicates the IMU-based respiratory rate is less reliable than the PPG-based respiratory rate; and
selecting the IMU-based respiratory rate as the final respiratory rate where the quality metric for the IMU-based respiratory rate is more reliable than the PPG-based respiratory rate.

13. The computer-implemented method of claim 8, further comprising:
filtering the sensor data for removal of Mayer-wave in-band noise, transients, and motion artifacts.

14. The computer-implemented method of claim 8, further comprising:
updating at least one threshold value associated with the set of rules by a machine learning model in real-time.

15. One or more computer storage devices having computer-executable instructions stored thereon, which, upon execution by a computer, cause the computer to perform operations comprising:
obtaining photoplethysmography (PPG) sensor data from a PPG sensor device and inertial movement unit (IMU) sensor data from an IMU sensor device;
applying a set of rules for filtering and processing the sensor data, wherein the set of rules comprises at least one rule for filtering out IMU signal data associated with motion activity unrelated to respiratory activity of a user;
generating respiratory rate estimates, including a PPG-based respiratory rate estimate based on the PPG sensor data and an IMU-based respiratory rate estimate based on the IMU sensor data in parallel;
selecting a final respiratory rate from the respiratory rate estimates based on a quality metric, wherein the quality metric comprises a quality score; and
providing the final respiratory rate and the quality score to a user via a user interface.

16. The one or more computer storage devices of claim 15, wherein the operations further comprise:
applying a peak-to-trough threshold, wherein a PPG signal having an amplitude less than the peak-to-trough threshold is filtered out as noise.

17. The one or more computer storage devices of claim 15, wherein the operations further comprise:
applying a motion activity threshold, wherein motion activity around an interval associated with an IMU-based respiratory signal less than a predetermined threshold value is used for calculating an estimated respiratory rate, and wherein motion activity around the interval greater than the predetermined threshold value is filtered out.

18. The one or more computer storage devices of claim 15, wherein the operations further comprise:
filtering the sensor data for removal of Mayer-wave in-band noise, transients, and motion artifacts.

19. The one or more computer storage devices of claim 15, wherein the IMU-based respiratory estimate comprises at least one of accelerometer-based respiratory rate estimate generated using sensor data from an accelerometer and gyroscope-based respiratory rate estimate generated based on sensor data generated by a gyroscope.

20. The one or more computer storage devices of claim 15, wherein the operations further comprise:
updating at least one rule in the set of rules by a respiratory rate performance optimization machine learning model in real-time.
